(19) United States
(12) Patent Application Publication
(10) Pub. No.: US 2023/0176057 A1
(43) Pub. Date: Jun. 8, 2023

(54) DETECTION ASSAY FOR SARS-COV-2 VIRUS

(71) Applicants: Chan Zuckerberg Biohub, Inc., San Francisco, CA (US); The Regents of the University of California, Oakland, CA (US)

(72) Inventors: James A. Wells, Oakland, CA (US); Susanna Elledge, Oakland, CA (US); Xin Zhou, Oakland, CA (US); Tanja Kortemme, Oakland, CA (US); Jeff Glasgow, Oakland, CA (US); Anum Glasgow, Oakland, CA (US); Irene Lui, Oakland, CA (US)

(21) Appl. No.: 17/997,875

(22) PCT Filed: May 11, 2021
(86) PCT No.: PCT/US2021/031850

§ 371 Date: Nov. 3, 2022

Related U.S. Application Data
(60) Provisional application No. 63/022,789, filed on May 11, 2020.
(60) Provisional application No. 63/056,509, filed on Jul. 24, 2020.
(60) Provisional application No. 63/058,379, filed on Jul. 29, 2020.
(60) Provisional application No. 63/067,273, filed on Aug. 18, 2020.

(51) Int. Cl. G01N 33/569; G01N 33/543; C07K 16/10

(52) U.S. Cl. CPC G01N 33/56983; C07K 16/10; G01N 33/54386; C07K 2319/00; G01N 2333/165

(57) ABSTRACT

Provided herein are protein biosensors, fusion proteins, compositions, and methods that are useful in detecting SARS-CoV-2 viruses in a sample from a subject. The viral detection assays described herein are solution-based, rapid, and quantitative. The protein biosensors and fusion proteins herein are able to bind to SARS-CoV-2 viral proteins. Use of the fusion proteins in proximity assays (e.g., split reporter assays) allows sensitive detection of SARS-CoV-2 virus in samples.

### CROSS-REFERENCES TO RELATED APPLICATIONS

This application claims priority to U.S. Provisional Application No. 63/022,789, filed on May 11, 2020; U.S. Provisional Application No. 63/056,509, filed on Jul. 24, 2020; U.S. Provisional Application No. 63/058,379, filed on Jul. 29, 2020; and U.S. Provisional Application No. 63/067,273, filed on Aug. 18, 2020. The entire disclosure of each of the aforementioned provisional applications is herein incorporated by reference for all purposes.

### STATEMENT AS TO RIGHTS TO INVENTIONS MADE UNDER FEDERALLY SPONSORED RESEARCH AND DEVELOPMENT

This invention was made with government support under grant no. K99 GM135529 awarded by The National Institutes of Health. The government has certain rights in the invention.

### SEQUENCE LISTING

The official copy of the sequence listing is submitted electronically via EFS-Web as an ASCII formatted sequence listing with a file named 1244642_seqlist.txt, created on May 11, 2021, and having a size of 199 KB, and is filed concurrently with the specification. The sequence listing contained in this ASCII formatted document is part of the specification and is herein incorporated by reference in its entirety.

### BACKGROUND

COVID-19, caused by the SARS-CoV-2 virus, has spread throughout the world and as of now has resulted in over 153 million cases and over 3.2 million deaths globally. Early detection of disease using viral detection assays is very critical to contain the spread of this virus. Clinical laboratory tests and point-of-care tests are needed for screening and diagnosis of infected individuals. The most widely used tests currently are PCR based tests that detect viral RNA in patient samples. See Esbin et al., 2020, “Overcoming the bottleneck to widespread testing: A rapid review of nucleic acid testing approaches for COVID-19 detection” RNA doi:10.1261/rna.076232.120. However, these methods require viral RNA extraction, reverse transcription PCR, and quantitative PCR reactions, which limit the throughput of the assay, requires expensive equipment and reagents, and takes hours or days to produce results. There is a need for sensitive, rapid tests for detecting SARS-CoV-2 virus in patient samples.

### BRIEF SUMMARY

In one aspect, provided herein are methods for detecting SARS-CoV virus in a test sample. In some embodiments, the methods comprise producing a mixture by combining (a) at least a portion of the test sample; (b) a first fusion protein that comprises a first viral protein-binding domain and either a first peptide fragment of a split reporter protein or a first reporter moiety; and (c) a second fusion protein that comprises a second viral protein-binding domain and either a second peptide fragment of the split reporter protein or a second reporter moiety. In some embodiments, the methods comprise maintaining the mixture under conditions in which, only if the test sample comprises SARS-CoV virus, the first peptide fragment and the second peptide fragment associate to produce an enzymatically active reporter protein or the first reporter moiety and the second reporter moiety specifically associate. In some embodiments, the methods comprise detecting the association of the first peptide fragment and the second peptide fragment or the first reporter moiety and the second reporter moiety if the test sample comprises SARS-CoV virus. In some embodiments, the SARS-CoV virus is SARS-CoV-2.

In some embodiments, the first viral protein-binding domain and the second viral protein-binding domain of the fusion proteins used in the methods described herein are each selected from the group consisting of an ACE2 polypeptide domain, a spike-binding antibody domain, and a nucleocapsid protein-binding antibody domain.

In some embodiments, each of the first viral protein-binding domain and the second viral protein-binding domain of the fusion proteins used in the methods described herein is an ACE2 polypeptide domain or a spike-binding antibody domain. In some embodiments, the first viral protein-binding domain and the second viral protein-binding domain both bind to a first spike protein binding site. In some embodiments, the first viral protein-binding domain binds to the first spike protein binding site and the second viral protein-binding domain binds to a second spike protein binding site. In some embodiments, the first spike protein binding site and/or the second spike protein binding site are within a spike protein receptor binding domain (RBD). In some embodiments, the first spike protein binding site and/or the second spike protein binding site are not within a spike protein RBD.

In some embodiments, each of the first viral protein-binding domain and the second viral protein-binding domain of the fusion proteins used in the methods described herein is a nucleocapsid protein-binding antibody domain. In some embodiments, the first viral protein-binding domain and the second viral protein-binding domain both bind to a first nucleocapsid protein binding site. In some embodiments, the first viral protein-binding domain binds to the first nucleocapsid protein binding site and the second viral protein-binding domain binds to a second nucleocapsid protein binding site.

In some embodiments, the fusion proteins used in the methods described herein comprise a dimerization domain. In some embodiments, the dimerization domain comprises an antibody Fc domain.

In some embodiments of the methods described herein, if the test sample comprises SARS-CoV virus, the first fusion protein binds to a first viral protein on a virion and the second fusion protein binds to the first viral protein or to a second viral protein on the same virion. In some embodiments, the first viral protein and the second viral protein are each selected from the group consisting of a spike protein and a nucleocapsid protein. In some embodiments, the mixture comprises detection reagents and a detectable signal is produced by the action of the enzymatically active reporter protein in the presence of the detection reagents. In some embodiments, the association of the first peptide fragment and the second peptide fragment to produce the enzymatically active reporter protein comprises association of the first peptide fragment, the second peptide fragment and a third peptide fragment of the reporter protein. In some embodiments, the reporter protein used in the methods described herein is luciferase.

In some embodiments of the methods described herein, the first reporter moiety and the second reporter moiety are oligonucleotides that are partially complementary to each other or are both partially complementary to an oligonucleotide in the mixture. In some embodiments, the mixture comprises detection reagents and a detectable signal is produced by the specific association of the first and second reporter moieties in the presence of the detection reagents.

In some embodiments of the methods provided herein, the SARS-CoV virus being detected is SARS-CoV-2. In some embodiments, SARS-CoV-2 is detected at a concentration of less than 1×10⁸ viral particles per mL.

In another aspect, provided herein are fusion proteins that comprise a viral protein-binding domain and a peptide fragment of a reporter protein or a first reporter moiety. In some embodiments, the fusion proteins comprise an RBD-binding ACE2 polypeptide domain and a first peptide fragment of a split reporter protein or a first reporter moiety. In some embodiments, the fusion proteins comprise a spike-binding antibody domain and a peptide fragment of a split reporter protein or a reporter moiety. In some embodiments, the fusion proteins comprise a nucleocapsid protein-binding antibody domain and a peptide fragment of a split reporter protein or a reporter moiety. In some embodiments, any of the fusion proteins provided herein comprise a dimerization domain (e.g., an antibody Fc domain).

Also provided herein are compositions comprising two fusion proteins as described above. In some embodiments, the split reporter proteins of the two fusion proteins are complementary fragments of a reporter protein. In some embodiments, the reporter moieties of the two fusion proteins are oligonucleotides that are partially complementary to each other or are both partially complementary to an additional oligonucleotide.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a schematic illustration of FL-Spike Trimer, Spike-RBD-Fc, Spike-RBD monomer, ACE2-Fc and ACE2 monomer, according to aspects of this disclosure. A panel of Spike and ACE2 proteins in various multimeric formats were constructed. FL-Spike trimer (top row, left panel) includes 3 subunits of SARS-CoV-2 FL-Spike ectodomain (aa 1-1213), each of which includes an S2 domain, an S1 domain, and a receptor binding domain (RBD). Additionally, a T4 trimerization motif and His6 tag were added to the C-terminus. Spike-RBD-Fc (top row, middle panel) includes two subunits of SARS-CoV-2 Spike RBD linked to a human Fc domain via a TEV-cleavable linker. Each subunit also includes a C-terminal AviTag™ for biotinylation. Spike-RBD monomer (top row, right panel) includes a C-terminal TEV recognition site-His8 tag-AviTag™ ACE2-Fc (bottom row, left panel) includes two subunits of human ACE2 signal sequence and peptidase domain (amino acids 1-614) linked to a human Fc domain via a TEV-cleavable linker. Each subunit also includes a C-terminal AviTag™ for biotinylation. ACE2 monomer (bottom row, right panel) was generated by cleavage of ACE2-Fc using TEV protease.

FIG. 2 shows characterization of expressed and purified Spike and ACE2 variant proteins shown in FIG. 1, according to aspects of this disclosure. The sizes of the proteins were estimated using size exclusion chromatography (SEC). The plots show SEC traces of each purified protein. Dotted lines mark retention time of a molecular weight standard.

FIG. 3 shows ACE2-Fc split luciferase experiments demonstrating that ACE2 binds to FL-Spike RBD domains, according to aspects of this disclosure. The top panel shows a schematic depiction of the N-terminal ACE2 NanoBiT sensor system (i.e., SmBiT-ACE2-Fc and LgBiT-ACE2-Fc) used to detect SpikeRBD-Fc bound to streptavidin magnetic beads. Binding of both ACE2 fusion proteins to nearby bead-bound SpikeRBD-Fc leads to reconstitution of the luciferase protein (NanoBiT) and generation of luminescence. The bottom panel is a graph showing quantification of luminescence in the presence of increasing amounts of FL-Spike bound to the beads.

FIG. 4 shows ACE2-Fc split luciferase experiments demonstrating that more than one RBD in FL-Spike may be available to bind ACE2, according to aspects of this disclosure. The top panel shows a schematic depiction of the N-terminal ACE2 NanoBiT sensor system (i.e., SmBiT-ACE2-Fc and LgBiT-ACE2-Fc) used to detect SpikeRBD-Fc in solution. Binding of both ACE2 fusion proteins to RBD domains in a FL-Spike trimer leads to reconstitution of the luciferase protein (NanoBiT) and generation of luminescence. The bottom panel is a graph showing quantification of luminescence in the presence of increasing amounts of FL-Spike in solution. The average is plotted for N=3 samples and the error bars represent the standard deviation.

FIG. 5 shows a schematic depiction of a C-terminal ACE2 NanoBiT sensor system (i.e., ACE2-Fc-SmBiT and ACE2-Fc-LgBiT) used to detect SpikeRBD-Fc bound to streptavidin beads. Binding of both ACE2 fusion proteins to nearby bead-bound SpikeRBD-Fc leads to reconstitution of the luciferase protein (NanoBiT) and generation of luminescence.

FIG. 6 shows a schematic depiction of a C-terminal ACE2 NanoBiT sensor system (i.e., ACE2-Fc-SmBiT and ACE2-Fc-LgBiT) used to detect SpikeRBD-Fc in solution. Binding of both ACE2 fusion proteins to RBD domains in a FL-Spike trimer leads to reconstitution of the luciferase protein (NanoBiT) and generation of luminescence.

FIG. 7 shows a schematic illustration of a NanoBiT sensor system with ACE2-Fc-LgBiT in combination with ACE2-Fc-SmBiT that can be used to detect two FL-Spike Trimers on a viral surface according to aspects of this disclosure.

FIG. 8 shows that the an ACE2-Fc NanoBiT system is able to detect various concentrations of Spike-RBD-Fc immobilized on a magnetic bead surface (used as a mimetic of Spike present on virus surface) through a luminescence readout according to aspects of this disclosure.

FIG. 9 (top panel) shows a schematic illustration of one of the NanoBiT sensor systems with ACE2-Fc-LgBiT in combination with IgG-SmBiT that can be used to detect FL-Spike Trimer according to aspects of this disclosure. FIG. 9 (bottom panel) shows the ACE2-Antibody NanoBiT system is able to detect various concentrations of FL-Spike in solution through a luminescence readout according to aspects of this disclosure.

FIG. 10 shows that the ACE2-Fc-LgBiT and IgG-SmBiT sensor system (depicted in FIG. 9, top panel) can detect pseudotyped lentivirus expressing SARS-CoV-2 Spike glycoprotein according to aspects of this disclosure.

FIG. 11 shows schematic illustrations of exemplary constructs used for the split reporter assays according to aspects of this disclosure. The short name for each construct as used in the Examples is located above the corresponding schematic illustration.

FIG. 12 shows that a Fab fragment of nucleocapsid-binding antibody clone H2 binds the SARS-CoV-2 nucleocapsid protein with a K_{D} of 20 nM, according to aspects of this disclosure. SARS-CoV-2 nucleocapsid protein was immobilized on a streptavidin sensor and incubated with various concentrations of the clone H2 Fab fragment (concentrations used are 200 nM, 100 nM, 50 nM, 25 nM, 12.5 nM, 6.25 nM, and 3.125 nM) in a bio-layer interferometry experiment. Shown are binding/dissociation curves for the nucleocapsid protein and the H2 Fab fragment at decreasing concentrations from top to bottom.

FIG. 13 shows that the nucleocapsid (N) protein antibody (H2 clone), in either Fab or scFv format, linked to LgBiT and SmBiT detected SARS-CoV-2 N protein with a limit of detection (LOD) of~1.9 ng/mL, according to aspects of this disclosure. Both protein biosensors (i.e., H2 Fab-SmBiT/H2 Fab-LgBiT and H2 LC-HC scFv-SmBiT/H2 LC-HC scFv-LgBiT) were used at 1 nM to detection various concentrations of the N protein. The H2 Fab/scFv sensors were incubated with the N protein for 20 minutes at room temperature followed by addition of NanoLuc substrates, 10-minute incubation, and readout on a luminometer for 1 second. Shown are the relative luciferase unit (RLU) read-outs of NanoLuc (NLuc) activity for the H2 Fab protein biosensor (top panel) and H2 scFv protein biosensor (bottom panel). The H2 Fab protein biosensors were also incubated with increasing concentrations of heat inactivated (65° C. for 30 min) N protein (square points in top panel) and showed similar performance to non-heat inactivated (“Fresh,” circle points in top panel). The Fab version of the sensors were tested against both the fresh and the heat inactivated proteins (65c 30 min) and showed similar performance.

### DETAILED DESCRIPTION

### I. Introduction

Provided herein are protein biosensors, fusion proteins, compositions, and methods for detecting SARS-CoV-2 virus in a solution-based, rapid, and quantitative viral detection assay. This section describes certain general feature of protein biosensors, but is not intended to be limiting or comprehensive.

A “protein biosensor,” as used herein, may refer to a pair of fusion proteins (which may be called a cognate pair) that can be used together to detect SARS-CoV-2 virus (e.g., by detecting SARS-CoV-2 proteins). Each fusion protein of the pair comprises a viral protein-binding domain (V domain) and a detection moiety, where the detection moieties of the two members of the pair are complementary portions of a split reporter. As used in this context, “complementary” means that, when in proximity, the detection moieties (optionally with other components) may combine to generate a detectable complex. In general, the split reporter fragments have low affinity for one another, and the split reporter is only detectable when its at least two parts are reconstituted. Classic split reporters are proteins, typically enzymes, that become fully functional following the interaction of two or more protein fragments that have no activity on their own. For example, split polypeptide detection moieties may combine to form a complex with a luciferase activity not found in either individual moiety. In the context of this disclosure, the term split reporter is also used to refer to two or more split oligonucleotide detection moieties that may hybridize to each other, or to a common splint oligonucleotide, to form a nucleic acid complex that can be detected, e.g., by ligation, extension, and/or amplification of the nucleic acid complex. Assays using split oligonucleotides include proximity extension assays and proximity ligation assays. In some instances, the detection moiety may be another detectable moiety (e.g., a chemical functional group, a fluorophore, biotin).

In some embodiments the V domain and the detection moiety (D) are connected by a peptide linker domain (L). Thus, for illustration and not limitation in some embodiments each member of a construct pair is a fusion protein with the structure V-D or V-L-D. Optionally additional sequences may be found (e.g., amino-terminal to V). In some embodiments, when the two members of a construct pair bind to the viral protein(s) (e.g., the two members bind to the same viral protein or the two members bind to two viral proteins on the same virus particle), the detection moieties are brought into proximity and associate to form an active reporter. In this disclosure, when the reporter is a protein, a detection moiety may alternatively be referred to as a “detection moiety domain” or a “peptide fragment.” In this example, a signal generated from the active reporter protein can be quantified to indicate the presence of the antibody.

For convenience, the two fusion protein members of a cognate pair can be referred to as alpha (α) and beta (β). For illustration and not for limitation, the structure of a first member can be described as αV-αD or αV-αL-αD and the structure of the second member can be described as βV-βD or βV-βL-βD. As noted, αD and βD are complementary portions of a split reporter.

In some embodiments the viral protein-binding domains αV and βV are the same (i.e., have identical amino acid sequences) reflecting that each binds the same viral protein. However, it is contemplated that in some embodiments αV and βV may have different amino acid sequences, provided each amino acid sequence is able to bind a SARS-CoV-2 viral protein. In some embodiments, αV and βV, if not identical, will have similar sequences.

The linker moieties, αL and βL may be the same or different (e.g., the αL and βL may be of different lengths or sequences). Both, only one, or neither of αL and βL may be present in the fusion protein pair. More discussion of linker moieties is included below.

As discussed in detail below, in one aspect, the viral protein-binding domains of the fusion proteins provided herein are from an antibody that binds a SARS-CoV-2 viral protein (e.g., the SARS-CoV-2 nucleocapsid (N) protein, the SARS-CoV-2 Spike (S) protein, or the RBD portion of the SARS-CoV-2 S protein).

It will be recognized by the skilled reader that, although the protein biosensors provided herein detect SARS-CoV-2 N or S proteins, the protein biosensors may be adapted for detecting other proteins from the SARS-CoV-2 virus, as well as a broad range of other viral or bacterial proteins associated with other infectious diseases (e.g., the SARS-CoV-1 N or S protein).

The terms “protein biosensor,” “viral protein biosensor,” “sensor,” “biosensor” and the like are used interchangeably.

### II. Terminology

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Coronaviruses are a group of enveloped, single-stranded RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. The following strains of human coronaviruses are currently known: Human coronavirus 229E (HCoV-229E); Human coronavirus OC43 (HCoV-OC43); Severe acute respiratory syndrome coronavirus (SARS-CoV or SARS-CoV-1); Human coronavirus NL63 (HCoV-NL63, New Haven coronavirus); Human coronavirus HKU1 (HCoV-HKU1), which originated from infected mice, was first discovered in January 2005 in two patients in Hong Kong; Middle East respiratory syndrome-related coronavirus (MERS-CoV), also known as novel coronavirus 2012 and HCoV-EMC; and Severe acute respiratory syndrome coronavirus 2 (SARS-CoV-2), also known as 2019-nCoV or “novel coronavirus 2019.” Several variants of SARS-CoV-2 have been identified, including B.1.1.7, also known as the “UK variant,” initially detected in the United Kingdom, and B.1.351, also known as the “South Africa variant,” initially detected in South Africa in December 2020. The coronaviruses HCoV-229E, -NL63, -OC43, and -HKU1 continually circulate in the human population and cause respiratory infections in adults and children world-wide.

“Virus” is used in both the plural and singular senses. “Virion” refers to a single infectious particle.

“SARS-CoV virus” or “virus” when used without modifiers, refers to SARS-CoV-2 virus. However, it will be understood that the methods described herein, including assays for virus, viral protein biosensors, and anti-virus antibodies can be used to detect other coronaviruses (e.g., SARS-CoV-1 virus).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The terms “nucleic acid”, “polynucleotide” and “oligonucleotide,” as well as the related terms, interchangeably refer to DNA, RNA, and polymers thereof in single-stranded, double-stranded, or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. . Unless specifically limited, the term encompasses nucleic acids containing known analogs of natural nucleotides that have similar binding properties as the reference nucleic acid.

“Fusion protein” refers to a protein comprising two different polypeptide sequences, i.e. a first domain and a second domain, that are joined or linked to form a single polypeptide. The two amino acid sequences are encoded by separate nucleic acid sequences that have been joined so that they are transcribed and translated to produce a single polypeptide. The two domains can be contiguous, separated by one or more spacer, linker or hinge sequences, or separated by an additional polypeptide domain. An “Fc-fusion protein” includes an Fc domain (i.e., a monomer corresponding to an Fc homodimer). An “IgG-fusion protein” includes an IgG domain. An “ACE2-fusion protein” includes an ACE2 domain. “Fusion protein” also refers to a protein comprising a polypeptide sequence linked to a non-protein moiety (e.g., an oligonucleotide, a fluorophore, or a chemical functional group).

A “domain” of a protein refers to a region of the protein defined by an amino acid sequence and/or a functional property. Functional properties include enzymatic activity and/or the ability to bind to or be bound by another protein or nonprotein entity.

A “protein dimer” has its normal meaning in the art and refers to a protein complex formed by two protein monomers, or single proteins, which are usually non-covalently bound.

The term “antibody” includes tetrameric antibodies, single chain antibodies, binding fragments of antibodies (Fab, Fab′, F(ab′)2, scFv, dsFv, ds-scFv) minibodies, bispecific antibodies, nanobodies, diabodies. See, Siontorou CG. 2013, “Nanobodies as novel agents for disease diagnosis and therapy,” Int J Nanomedicine 8:4215-4227. A natural immuoglobulin G (IgG) antibody molecule is a tetramer that contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. Within each light or heavy chain variable region, there are three short segments (averaging 10 amino acids in length) called the complementarity determining regions (“CDRs”). Light chains are classified as either kappa or lambda. Heavy chains are classified as gamma, mu, alpha, delta, or epsilon, and define the antibody’s isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. See generally, Fundamental Immunology, Paul, W., ed., 3rd ed. Raven Press, NY, 1993, SH. 9 (incorporated by reference in its entirety for all purposes). Antibody sequences and structural information is widely available. See, e.g., Lima et al., 2020, “The ABCD database: a repository for chemically defined antibodies” Nucleic Acids Research 48:D261-D264. An antibody digested by papain yields three fragments: two Fab fragments and one Fc fragment. The Fc fragment is dimeric and contains two CH2 and two CH3 heavy chain domains. CH3 domains interact to form a homodimer. See Yang et al., 2018, “Engineering of Fc Fragments with Optimized Physicochemical Properties Implying Improvement of Clinical Potentials for Fc-Based Therapeutics” Frontiers in Immunology 8:1860. Antibodies used in the methods described herein may have sequences derived from non-human antibodies, human sequence, chimeric sequences, and wholly synthetic sequences. Additional details of antibodies useful in the context of this disclosure are provided below.

A “Fc fragment” contains two heavy chain fragments comprising the CH2 and CH3 domains of an antibody. The two heavy chain fragments are held together by two or more disulfide bonds and by hydrophobic interactions of the CH3 domains. A Fc domain introduced into a fusion protein may promote dimerization.

A “Fab fragment” is comprised of one light chain, and the CH1 and variable regions of one heavy chain and can specifically recognize a target epitope, such as an epitope of a Spike protein. A Fab domain introduced into a fusion protein results in binding of the fusion protein to the target.

A “single-chain variable fragment” or “scFv fragment” is a fusion protein comprising the variable regions of a heavy chain and a light chain from an antibody. The heavy chain and light chain portions may be connected by a linker peptide. An scFv fragment may retain the binding specificity of the antibody from which it is derived.

### III. Subjects and Patient Samples

Provided herein are detection assays for detecting SARS-CoV virus using the fusion proteins described herein. In some embodiments, the assays are carried out by combining a sample (e.g., a sample from a patient or subject) with the fusion proteins.

As used herein, the term “subject” or “patient” refers to a mammalian subject. Exemplary subjects include, but are not limited to humans, monkeys, dogs, cats, mice, rats, cows, pigs, birds, horses, camels, goats, and sheep.

The terms “sample” and “test sample” refer to a material or composition tested for virus content. A sample may be a biological sample, a patient sample, a veterinary sample, an agricultural or food sample, an environmental (e.g., water) sample.

SARS-CoV-2 virus in a subject can be detected using the assays herein on a biological sample from the subject. The term “biological sample” refers to a sample from a subject that is tested for the presence of virus (including without limitation including a throat swab, a nasopharyngeal swab, a sputum or tracheal aspirate, a nasal aspirate, blood, serum, plasma, tissue, urine, or stool). A sample or patient sample may be processed prior to an assay including by dilution, addition of buffer or preservative, concentration, purification, or partial purification. In some instances, SARS-CoV-1 virus can be detected in a biological sample from a subject.

Typically a biological sample obtained from a human subject is referred to as a “patient sample.” The word “patient,” in this context does not connote that the subject is ill, infected, recovering from infection, or previously infected.

In some embodiments, the sample may be from a subject that is asymptomatic or symptomatic. The subject may be male or female and may be a juvenile or an adult (e.g., at least 30 years old, at least 40 years old, or at least 50 years old). In some embodiments, the subject is displaying one or more symptoms indicative of SARS-CoV-2 infection (i.e. of COVID-19). Such symptoms include, but are not limited to, any of a new loss of taste or smell, myalgia, fatigue, shortness of breath or difficulty breathing, fever, and/or cough. Symptoms may also include pharyngitis, headache, productive cough (i.e. a cough that produces mucus or phlegm), gastrointestinal symptoms (e.g., diarrhea, nausea, vomiting, or abdominal pain), hemoptysis, chest pressure or pain, confusion, cyanosis, and/or chills. In some embodiments, the patient has at least two symptoms selected from the group consisting of a new loss of taste or smell, shortness of breath or difficulty breathing, fever, cough, chills, or muscle aches. In some embodiments, the patient may have a blood oxygen level reading of 94 or less, e.g., as determined by an oximeter. In some embodiments, the subject may have radiographic evidence of pulmonary infiltrates. In some embodiments, the subject may have been receiving standard support care, e.g., such as being administered oxygen, fluids, and/or other therapeutic procedures or agents.

In some embodiments, the subject may not manifest any symptoms that are typically associated with SARS-CoV-2 infection. In some cases the subject is known or believed to have been exposed to SARS-CoV-2, suspected of having exposure to SARS-CoV-2, or believed not to have had exposure to SARS-CoV-2. In some cases, the subject may have recovered from a prior exposure of SARS-CoV-2. In some cases, the subject has received a SARS-CoV-2 vaccine. The SARS-CoV-2 vaccine can be any of the DNA, RNA, or protein, or inactive SARS-CoV-2 virus that is capable of inducing immune response in a patient to generate anti SARS-CoV-2 antibodies. In some cases, the subject has been free of symptoms suggestive of a SARS-CoV-2 infection for at least 14 days. In some cases, the subject may have one or more of other conditions of hypertension, coronary artery disease, diabetes, chronic obstructive pulmonary disease.

### IV. Protein Biosensors

Disclosed herein are protein biosensors based, in part, on the discoveries described in the Examples and discussed below, that are useful in proximity-based binding assays for the detection of SARS-CoV-2 virus (e.g., by detection of a SARS-CoV-2 viral protein) in a test sample. A proximity assay (or proximity-based binding assay) produces a detectable signal when two binding events occur physically close to each other and at the same time. Examples of proximity assays include split reporter-type assays, proximity ligation, and proximity extension assays.

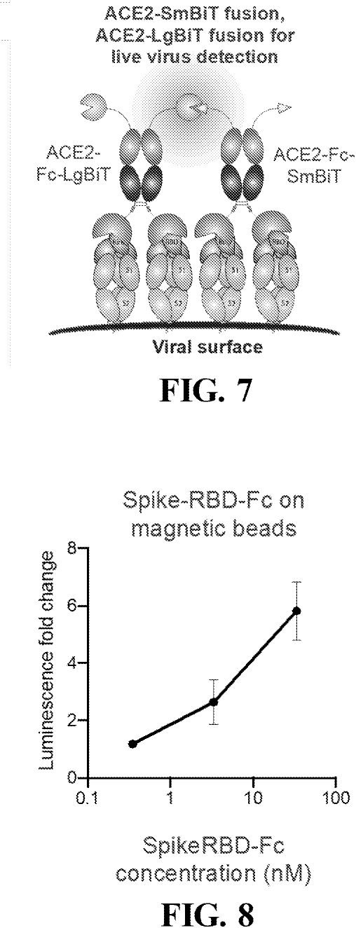

In some aspects, the assay involves combining a portion of the test sample (e.g., serum) with a protein sensor, e.g., a cognate pair of fusion proteins that detect one or more SARS-CoV-2 viral proteins, under conditions in which protein sensor-viral protein binding occurs if virus particles are present in the sample (“assay conditions”). As discussed above, in one aspect, a protein sensor includes a cognate pair of fusion proteins and each member of the pair comprises a viral protein-binding domain (V domain) fused to a detection moiety (e.g., a D domain), where the detection moieties of the two members are complementary portions of a split reporter. Stated differently, in one approach each protein sensor comprises a first fusion protein and a second fusion protein. The first fusion protein comprises a first viral protein-binding domain fused to a first detection moiety (e.g., a first peptide fragment of a split reporter protein or a first oligonucleotide of a split nucleic acid reporter complex). The second fusion protein comprises a second viral protein-binding domain fused to a second detection moiety (e.g., a second peptide fragment of the split reporter protein or a second oligonucleotide of the split nucleic acid reporter complex). In some embodiments, the sensor produces a detectable signal when virus is present in the patient sample, which brings the first detection moiety and the second detection moiety of the split reporter within proximity to each other. An example of a protein biosensor producing signal upon binding to viral proteins according to certain embodiments is shown in FIG. 7 and described in Example 6.

### A. Viral Protein-Binding Domains (V)

SARS-CoV-2 comprises a positive-strand RNA genome that encodes 16 non-structural proteins, nine accessory factors, and four structural proteins (S, E, M, and N) (Gordon et al., 2020, “Comparative host-coronavirus protein interaction networks reveal pan-viral disease mechanisms,” Science 370(6521):eabe9403) as well as accessory proteins with mostly unknown function (Narayanan et al., 2008, “SARS coronavirus accessory proteins,” Virus Res. 133(1):113-121). The viral protein-binding domains (V domains) provided herein may bind to any of the proteins or factors encoded by the SARS-CoV-2 genome. In some embodiments, the V domains bind to the Spike (S) protein. In some embodiments, the V domains bind to the Nucleocapsid (N) protein. In some embodiments, the V domains of a protein biosensor (αV, βV) include recombinant ACE2 polypeptides. In some embodiments, the V domains of a protein biosensor (αV, βV) include sequences from antibodies that bind a SARS-CoV-2 viral protein (e.g., the S protein or the N protein).

“Spike” proteins are coronavirus surface proteins that are able to mediate receptor binding and membrane fusion between the virus and host cell. Spikes are homotrimers of the S protein, which has S1 and S2 domains. In addition to mediating virus entry, the spike is an important determinant of viral host range and tissue tropism and a major inducer of host immune responses. The interaction between the SARS-CoV-2 Spike protein and the angiotensin-converting enzyme 2 (ACE2; described below) on human cells is critical for viral entry into host cells (Gralinski & Menachery, 2020, “Return of the coronavirus: 2019-nCoV,” Viruses 12(2):135; Tai et al., 2020, “Characterization of the receptor-binding domain (RBD) of 2019 novel coronavirus: implication for development of RBD protein as a viral attachment inhibitor and vaccine,” Cell. and Mol. Immun. 17(6):613-620; and Wu et al., 2020, “A new coronavirus associated with human respiratory disease in China,” Nature, 579(7798), 265-269). The receptor binding domain (RBD) is located on the S1 subunit and can bind to the receptor on target cells. See Walls et al., 2020, “Structure, function and antigenicity of the SARS-CoV-2 spike glycoprotein” Cell 181:281. An exemplary SARS-CoV-2 Spike RBD protein sequence is the amino acid sequence set forth in SEQ ID NO:9.

N protein, also called nucleocapsid protein N, packages the viral genome into a ribonucleocapsid and plays a fundamental role during viral self-assembly. See Chang et al, 2014, “The SARS coronavirus nucleocapsid protein--forms and functions,” Antiviral Res 103:39-50 and Zamecnik et al., 2020, “ReScan, a multiplex diagnostic pipeline, pans human sera for SARS-CoV-2 antigens,” Cell Reports Med. 1:100123. The N protein comprises an N-terminal RNA binding domain, which aids in viral RNA assembly and packaging into the viral particle. The N protein comprises a C-terminal dimerization domain (amino acid residues 258-419; SEQ ID NO: 10) and an RNA binding domain (amino acid residues 44-180; SEQ ID NO:11).

In some instances, the viral protein-binding domains provided herein bind to viral proteins expressed by SARS-CoV-1 (e.g., spike protein, nucleocapsid protein). In some instances, the viral protein-binding domains provided herein bind both to viral proteins expressed by SARS-CoV-1 and viral proteins expressed by SARS-CoV-2. In some instances, the viral protein-binding domains provided herein bind preferentially to viral proteins expressed by SARS-CoV-1 or SARS-CoV-2. In some instances, the viral protein-binding domains provided herein bind preferentially to viral proteins expressed by SARS-CoV-2. For convenience, discussion herein generally refers only to SARS-CoV-2 proteins.

### ACE2 Polypeptide V Domains

Full-length human ACE2 is 805 amino acids in length (SEQ ID NO: 1), of which amino acids 1-17 is a signal peptide that is cleaved from the mature protein. See NCBI Reference Sequence NP_001358344.1; see also UniProtKB Reference Q9BYF1. The ACE2 ectodomain (SEQ ID NO: 2) is composed of a N-terminal peptidase domain (aa 18-614) (SEQ ID NO:3) and a C-terminal dimerization domain, also referred to as a “collectrin” domain (aa 615-740) (SEQ ID NO:4). Recent studies have revealed the structural basis of the high-affinity ACE2-spike interaction through the spike receptor binding domain (RBD) (Lan, J., et al., Nature, 581:215-220 (2020) and Yan, R., et al., Science, 367(6485):1444-1448 (2020)). The ACE2-RBD co-structure shows a large, flat binding interface primarily comprising the N-terminal helices of ACE2 (residues 18-90), with secondary interaction sites spanning residues 324-361. It has also been determined that binding affinity of the ACE2-spike interaction is further improved through intermolecular avidity effects, as demonstrated by the efficacy of engineered dimeric ACE2-Fc fusion proteins in neutralizing SARS-CoV-2. See U.S. Provisional Pat. Application 63/022,789, Lui, I., et al., 2020, “Trimeric SARS-CoV-2 Spike interacts with dimeric ACE2 with limited intra-Spike avidity,” bioRxiv, published May 21, 2020, doi:10.1101/2020.05.21.109157 (referred to as “Lui et al., 2020” throughout this disclosure), and Glasgow et al., 2020, “Engineered ACE2 receptor traps potently neutralize SARS-CoV-2,” Proc. Nat. Acad. Sci. 117(45):28046-28055 (referred to as “Glasgow et al., 2020” throughout this disclosure), all three of which are incorporated herein in their entireties for all purposes.

In some embodiments, the viral protein-binding domain (V domain) of the protein sensors provided herein comprises an ACE2 polypeptide. In some embodiments, the ACE2 polypeptide has a wild-type sequence. Exemplary wild-type ACE2 sequences are provided as SEQ ID NO:4 (1-614), SEQ ID NO:3 (18-614), SEQ ID NO:1 (full-length 805), SEQ ID NO:6 (1-740), and SEQ ID NO:2 (18-740). In some embodiments, the V domain comprises fragments and/or variants of the wild-type ACE2 sequence. In some embodiments, the fragments are at least 596 amino acids in length.

In some embodiments the ACE2 polypeptide has the sequence of SEQ ID NO:3 (18-614). In one approach the ACE2 domain has the sequence of an ACE2 variant with a sequence that is substantially identical to SEQ ID NO:3, provided the variant binds the spike RBD (SEQ ID NO:9). In some embodiments, for example, the ACE2 variant may have at least 80%, or at least 90%, or at least 95% amino acid residue identity SEQ ID NO:3. In one approach the ACE2 domain is a fragment of SEQ ID NO:3 or a fragment of a variant of SEQ ID NO:3, provided the fragment binds the RBD. In some cases the variant or fragment is at least 300 residues in length, at least 400 residues in length, at least 500 residues in length, at least 550 residues in length or at least 600 residues in length.

In some embodiments, the V domain comprises a recombinant ACE2 polypeptide with one or more amino acid residue substitutions that result in increased or substantially increased binding affinity for the spike RBD compared to wild-type ACE2, in some instances 170 fold greater binding or more. The recombinant ACE2 polypeptides are variants of the ACE2 ectodomain and have improved binding affinity for the SARS-CoV-2 spike RBD as compared to wild-type ACE2 ectodomain. In some embodiments, the recombinant ACE2 polypeptides have improved binding affinity for the spike RBD from the B.1.1.7 and/or B.1.351 SARS-CoV-2 variants as compared to wild-type ACE2 ectodomain.

In some embodiments, the recombinant ACE2 polypeptides comprise a soluble ACE2 receptor ectodomain polypeptide comprising an amino acid sequence having at least 80% sequence identity (e.g., at least 90%, at least 95%) to SEQ ID NO: 2 or 3 and comprising at least one of the following amino acid residue substitutions: Q18R, S19P, A25V, T27A, T27Y, K31F, K31Y, N33D, N33S, H34A, H34I, H34S, H34V, E35Q, F40D, F40L, F40S, Q42L, N49D, N49S, N51S, N53S, E57G, N61D, M62T, M62I, M62V, N64D, K68R, W69R, W69V, W69K, W69I, Q76R, L79P, L79F, L79T, N90Q, L91P, L100P, Q101R, wherein the residues are numbered with reference to SEQ ID NO:1. In some instances, the recombinant ACE2 polypeptides may include at least two of these amino acid residue substitutions.

In some embodiments, the recombinant ACE2 polypeptides comprise a soluble ACE2 receptor ectodomain polypeptide comprising an amino acid sequence having at least 80% sequence identity (e.g., at least 90%) to SEQ ID NO:2, which includes an ACE2 collectrin domain (SEQ ID NO:4). In the full length ACE2 protein, the collectrin domain connects the extracellular domain of ACE2 to its transmembrane helix. The collectrin domain may stabilize the soluble extracellular part of the protein as a dimer through inter-collectrin domain contacts as well as additional C-terminal contacts between peptidase domains. In some embodiments, the collectrin domain facilitates dimerization of two ACE2 polypeptides to form a dimer. In some embodiments, recombinant ACE2 polypeptides comprising the collectrin domain have increased affinity for the spike RBD over polypeptides that do not comprise the collectrin domain, as described, for example, in Glasgow et al., 2020. In some embodiments, the recombinant ACE2 polypeptides comprise an amino acid sequence having at least 80% sequence identity (e.g., at least 90%) to SEQ ID NO:3, which does not include an ACE2 collectrin domain.

In some embodiments, the recombinant ACE2 polypeptides comprise a soluble ACE2 receptor ectodomain polypeptide comprising an amino acid sequence having at least 80% sequence identity (e.g., at least 90%) to SEQ ID NO: 2 or 3 and comprising amino acid residue substitutions in at least one of the following combinations:
i. K31F, N33D, H34S, and E35Q;
ii. K31F, N33D, H34A, E35Q, N49D, N51S, N53S, E57G, and N64D;
iii. T27A, K31F, N33D, H34S, E35Q, N61D, K68R, and L79P;
iv. S19P, N33S, H34V, F40L, N49D, and L100P;
v. K31F, N33D, H34S, E35Q, W69R, and Q76R;
vi. Q18R, K31F, N33D, H34S, E35Q, W69R, and Q76R;
vii. Q18R, K31F, N33D, H34S, E35Q, W69V, and Q76R;
viii. Q18R, K31F, N33D, H34S, E35Q, W69K, and Q76R;
ix. Q18R, K31F, N33D, H34S, E35Q, W69I, and Q76R;
x. T27A, H34A, N49S, V59A, N63S, K68R, E75G, N90Q, and Q103R;
xi. K31F, N33D, H34T, N53D, W69R, and E75K;
xii. S19P, K26R, T27A, H34A, S44G, and M62T;
xiii. K31F, H34I, E35Q, and N90Q;
xiv. A25V, T27A, H34A, and F40D;
xv. K31Y, W69V, L79T, and L91P;
xvi. T27Y, H34A, and N90Q;
xvii. S19P, Q42L, L79T, and N90Q;
xviii. K31F, H34I, E35Q; or
xix. H34V and N90Q,
wherein the residues are numbered with reference to SEQ ID NO: 1.

In some embodiments, the recombinant ACE2 polypeptides comprise a soluble ACE2 receptor polypeptide comprising an amino acid sequence having at least 80% sequence identity (e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO:7 or SEQ ID NO:8. In some embodiments, the recombinant ACE2 polypeptide comprises a soluble ACE2 receptor ectodomain polypeptide comprising an amino acid sequence having at least 80% sequence identity (e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to SEQ ID NO: 7 or SEQ ID NO:8 and comprising amino acid residue substitutions relative to SEQ ID NO: 1 in one or more of the following positions: K31F, N33D, H34S, and E35Q, wherein the residues are numbered with reference to SEQ ID NO: 1.

Also provided in this disclosure are V domains comprising recombinant ACE2 polypeptides comprising a soluble ACE2 receptor ectodomain polypeptide comprising mutations that inactivate the peptidase function of ACE2. In the body, the peptidase domain of ACE2 catalyzes the hydrolysis of angiotensin II (a vasoconstrictor peptide) into angiotensin (1-7) (a vasodilator). As such, inactivating mutations in the peptidase domain can prevent off-target effects in viral detection assays (e.g., by reducing interaction between the recombinant ACE2 polypeptides and angiotensin II in a test sample). In some aspects, the inactivating mutations do not impact affinity of the polypeptides comprising the mutations for the SARS-CoV-2 spike RBD. In some embodiments, the recombinant ACE2 polypeptides comprise amino acid residue substitutions H374N and H378N to inactivate the peptidase function. In some embodiments, the recombinant ACE2 polypeptides comprise the amino acid residue substitution H345L to inactivate the peptidase function.

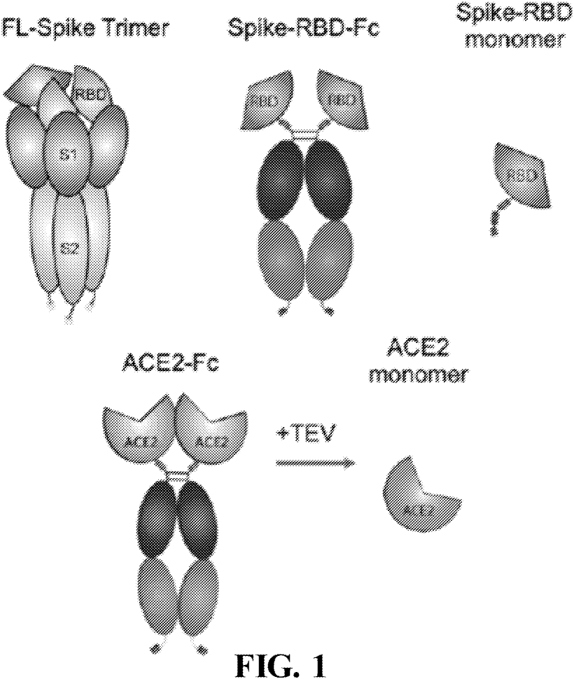

In some instances, the recombinant ACE2 polypeptides provided herein have increased binding affinity for monomeric SARS-CoV-2 spike RBD relative to wild-type human ACE2 protein ectodomain. As used herein, binding affinity of the provided recombinant ACE2 polypeptides and fusion proteins and monomeric SARS-CoV-2 spike RBD is measured as the dissociation constant “K_{D}” or apparent K_{D}. Binding affinity can be determined by a variety of methods known in the art. In some instances, bio-layer interferometry can be used to measure binding affinity. For example, as described in Examples 1 and 4 herein, in Glasgow et al., 2020, and in U.S. Provisional Pat. Application 63/022,789, the binding of ACE2 polypeptide variants or fusion proteins (e.g., the ACE2 proteins shown in FIG. 1) to full length spike protein or spike domains (e.g., the spike proteins shown in FIG. 1) can be measured by bio-layer interferometry. Bio-layer interferometry (“BLI”) is an optical technique for measuring macromolecular interactions by analyzing interference patterns of white light reflected from the surface of a biosensor tip coated with an immobilized protein, with any change in the number of molecules bound to the biosensor tip (i.e. protein-protein interactions) causing a shift in the interference pattern.

In some instances, binding affinity can be measured using titrations of purified monomeric spike RBD and yeast surface expressed recombinant ACE2 polypeptides as provided herein. The dissociation constant determined using yeast surface titrations is an estimate of the apparent K_{D} rather than the actual K_{D} due to the unknown multimerization state of the ACE2 molecule on the yeast cell surface. In some embodiments, the apparent K_{D} of the provided recombinant ACE2 polypeptides can be determined as described in Glasgow et al., 2020 using on-yeast protein display of variants lacking the collectrin domain with titrations of monomeric spike RBD.

In some instances, binding affinity can be measured by measuring the off rate of recombinant ACE2 polypeptides bound to spike protein or spike RBD in the presence of untagged inhibitor protein. In some embodiments, the binding affinity of a first recombinant ACE2 polypeptide for a spike protein or spike RBD measured using this method may be expressed 1) relative to the binding affinity of a second recombinant ACE2 polypeptide to the spike protein or spike RBD, or 2) relative to the binding affinity of the first recombinant ACE2 polypeptide to a different spike protein or spike RBD. For example, the binding affinity of a first recombinant ACE2 polypeptide for spike protein or spike RBD measured using this method may be expressed as within a factor of 2 (i.e., in the range of 2-fold weaker binding to 2-fold stronger binding) or within a factor of 3 (i.e., in the range of 3-fold weaker binding to 3-fold stronger binding) relative to the binding affinity of a second recombinant ACE2 polypeptide for spike protein or spike RBD or relative to the binding affinity of the first recombinant ACE2 polypeptide for a different spike protein or spike RBD. In some embodiments, a recombinant ACE2 polypeptide that has a binding affinity for spike protein or spike RBD measured using this method with a factor of 2 or within a factor of 3 relative to the binding affinity of the recombinant ACE2 polypeptide for a different spike protein or spike RBD may be said to have “similar binding” or to bind with a “similar off rate” to the different spike proteins or spike RBDs. The discussion above and the described embodiments also applies to assessment of bindingn affinity of antibody V domains, which are described below.

Other methods of measuring binding affinity include ELISA, surface plasmon resonance, or kinetic exclusion assays (Kinexa®). The K_{D} range in which measurements are accurate for different analytical methods may vary. For example, in some instances, as described in Examples 1, 2, and 5 of U.S. Provisional Application No. 63/058,379 and in Glasgow et al., 2020, the binding of ACE2 polypeptide variants that comprise the collectrin domain to full length spike protein may be too tight to be accurately measured by bio-layer interferometry. However, the apparent K_{D} can be measured for these variants using yeast surface titrations as described in Examples 1 and 3 of U.S. Provisional Application No. 63/058,379 and in Glasgow et al., 2020. One of skill in the art will appreciate that, within the accurate range, these methods will result in similar binding affinity measurements or similar trends in relative binding affinities for the various ACE2 polypeptides and fusion proteins described herein as compared to wild-type ACE2 and/or other ACE2 polypeptide variants.

In some instances, binding affinity for the recombinant ACE2 polypeptides and fusion proteins provided herein with monomeric SARS-CoV-2 spike RBD may be measured as an apparent K_{D} of less than 10 nM (for example, less than 9 nM, less than 7 nM, less than 5 nM, less than 4 nM, less than 3 nM, less than 2 nM, less than 1 nM, less than 0.5 nM, less than 0.25 nM, or less than 0.1 nM). Relative to wild-type human ACE2 protein ectodomain, the provided polypeptides and fusion proteins may have between 30-fold and 180-fold higher affinity for monomeric spike RBD (for example, between 40-fold and 160-fold, between 60-fold and 140-fold, between 80-fold and 120-fold, between 30-fold and 60-fold, between 150-fold and 180-fold, greather than 50-fold, greater than 100-fold, greater than 150-fold). In some instances, the provided polypeptides and fusion proteins may have greater than 180-fold higher affinity for monomeric SARS-CoV-2 spike RBD as compared to wild-type human ACE2 protein ectodomain. In one embodiment, a recombinant ACE2 fusion protein may have a binding affinity (apparent K_{D}) for monomeric spike RBD of 0.4 nM (51-fold higher than wild-type human ACE2 protein ectodomain; see e.g., variant 310 in Glasgow et al., 2020). In another embodiment, a recombinant ACE2 fusion protein may have a binding affinity (apparent K_{D}) for monomeric spike RBD of 0.64 nM (32-fold higher than wild-type human ACE2 protein ectodomain; see e.g., variant 311 in Glasgow et al., 2020). In one embodiment, a recombinant ACE2 fusion protein may have a binding affinity (apparent K_{D}) for monomeric spike RBD of 1.71 nM (12-fold higher than wild-type human ACE2 protein ectodomain; see e.g., variant 293 in Glasgow et al., 2020). In another embodiment, a recombinant ACE2 fusion protein may have a binding affinity (apparent K_{D}) for monomeric spike RBD of 0.52 nM (39-fold higher than wild-type human ACE2 protein ectodomain; see e.g., variant 313 in Glasgow et al., 2020). The wild-type human ACE2 protein ectodomain to which the binding affinity of the recombinant ACE2 polypeptides and fusion proteins is compared can have the amino acid sequence of SEQ ID NO: 2 or 3.

In some embodiments, the recombinant ACE2 polypeptides and fusion proteins provided herein show similar binding between 1) spike proteins or spike RBD from non-variant SARS-CoV-2 virus and 2) spike proteins or spike RBD from variant SARS-CoV-2 viruses (e.g., the B.1.1.7 UK variant and/or the B. 1.351 South Africa variant). In some embodiments, the recombinant ACE2 polypeptides bind to the UK variant and South Africa SARS-CoV-2 variant spike proteins with a similar off rate (i.e., within a factor of 3 or within a factor of 2) to the recombinant ACE2 polypeptides bound to non-variant SARS-CoV-2 spike protein (data not shown).

Natural ACE2 is pH sensitive and binds Spike 2-5X more tightly at pH 6.0 as compared to at pH 7.4, and this property is shared by some ACE2 variants (see FIG. 15 in U.S. Provisional Application No. 63/022,789). It is contemplated that in some implementations assays using protein biosensors comprising ACE2 polypeptides (e.g., according to the methods described below) are carried out at a pH less than 7.0 or less than 6.5 (e.g., pH 5-7, pH 5-6.5, or pH 5.5 to 6.5).

### Antibody V Domains

In some embodiments, the protein biosensors provided herein comprise V domains comprising antibody domains that bind to a SARS-CoV-2 viral protein (e.g., the Spike (S) protein or the nucleocapsid (N) protein). Thus, in some embodiments, the protein biosensors can be antibody fusion proteins. In some embodiments, the V domains comprise a spike-binding antibody. In some embodiments, the V domains comprise a nucleocapsid protein-binding antibody. In some embodiments, the antibody fusion proteins comprise an antibody domain and an Fc domain. In some embodiments, the antibody can be a single chain antibody (e.g., scFv), a tetrameric antibody or fragment thereof (e.g., Fab fragment, single-domain antibody, nanobody), a heavy chain sequence, and/or a light chain sequence. Also provided are antigen-binding fragments of any of the antibodies described herein. For example, the antigen-binding domain may be a spike-binding domain or a nucleocapid protein-binding domain.

Various methods for designing and expressing antibodies and antigen-binding fragments are known to those of skill in the art. For example, the pFUSE vectors available from Invitrogen offer a variety of antibody fusion protein formats (e.g., IgG and scFv formats), and expression of Fab fragments is described, e.g., in Hornsby et al., 2015, “A high through-put platform for recombinant antibodies to folded proteins,” Mol. Cell Proteomics 14(10):2833-2847. The antibodies, antibody fragments, and antibody domains provided herein may be designed, expressed, and purified using any suitable method, as described further below in Section V, “Nucleic acids, constructs, vectors and host cells” section. Antibodies and antibody domains are described in more detail below.

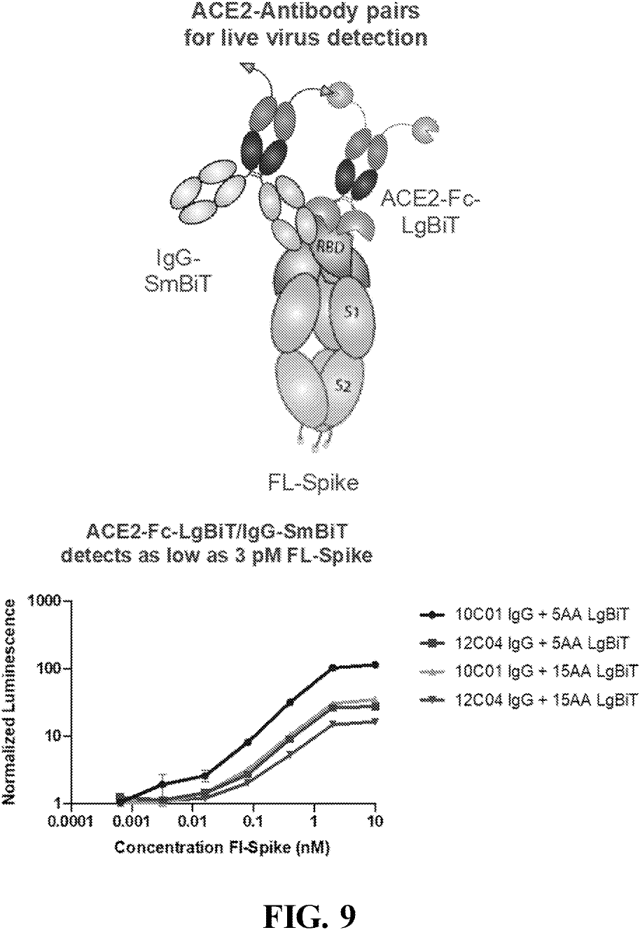

In some embodiments, the antigen-binding domain is a spike-binding antibody domain that binds an epitope of the Spike protein. In some embodiments, the antibody domain binds an epitope in the RBD of the Spike protein. In some embodiments, the antibody domain binds an epitope in the RBD without preventing interaction of the RBD with ACE2 (e.g., by binding a different sequence than is bound by ACE2; see, e.g., schematic depiction in FIG. 9, top panel). In some embodiments, the antibody domain binds a spike protein epitope that is not in the RBD.

In some embodiments, a spike-binding antibody domain comprises a light chain sequence having at least 90% identity (e.g., at least 91%, at least 92%, at least 93%, at least 94%, at least 95%, at least 96%, at least 97%, at least 98%, or at least 99%) to the amino acid sequence of SEQ ID NO: 12. In some embodiments, a spike-binding antibody domain comprises a heavy chain sequence having at least 90% identity (e.g., at least 91%, at least 92%, at least 93%, at least 94%, at least 95%, at least 96%, at least 97%, at least 98%, or at least 99%) to the amino acid sequence of SEQ ID NO: 13. In some embodiments, a spike-binding antibody domain comprises a light chain sequence having at least 90% identity (e.g., at least 91%, at least 92%, at least 93%, at least 94%, at least 95%, at least 96%, at least 97%, at least 98%, or at least 99%) to the amino acid sequence of SEQ ID NO: 12 and a heavy chain sequence having at least 90% identity (e.g., at least 91%, at least 92%, at least 93%, at least 94%, at least 95%, at least 96%, at least 97%, at least 98%, or at least 99%) to the amino acid sequence of SEQ ID NO: 13.

In some embodiments, a spike-binding antibody domain comprises a light chain variable region comprising (i) a CDRL1 comprising SEQ ID NO: 14; (ii) a CDRL2 comprising SEQ ID NO:15; and (iii) a CDRL3 comprising SEQ ID NO:16; and a heavy chain variable region comprising (i) a CDRH1 comprising SEQ ID NO: 17, a CDRH2 comprising SEQ ID NO: 18, and a CDRH3 comprising SEQ ID NO: 19. In some embodiments, a spike-binding antibody domain comprises at least one of the CDR sequences set forth in SEQ ID NOs: 14-19. In some embodiments, a spike-binding antibody domain comprises at least one of the CDRL sequences set forth in SEQ ID NOs: 14-16. In some embodiments, a spike-binding antibody domain comprises at least one of the CDRH sequences set forth in SEQ ID NOs: 17-19.

TABLE 1

Spike-binding antibody sequences

| | | | | | | | | |
|---|---|---|---|---|---|---|---|---|
| Antibody | Light chain | CDRL1 | CDRL2 | CDRL3 | Heavy chain | CDRH1 | CDRH2 | CDRH3 |
| Spike-binding | SEQ ID NO: 12 | SEQ ID NO: 14 | SEQ ID NO:15 | SEQ ID NO: 16 | SEQ ID NO: 13 | SEQ ID NO: 17 | SEQ ID NO:18 | SEQ ID NO: 19 |

Other spike-binding antibodies (including RBD-binding antibodies) are known including, for illustration and not limitation, those described in Yuan et al., 2020, “A highly conserved cryptic epitope in the receptor binding domains of SARS-CoV-2 and SARS-CoV,” Science 368:6491:630-633; Wrapp et al., 2020, “Structural Basis for Potent Neutralization of Betacoronaviruses by Single-Domain Camelid Antibodies,” Cell 118:1-12; Walter et al., 2020, “Synthetic nanobodies targeting the SARS-CoV-2 receptor-binding domain,” bioRxiv, Apr. 18, 2020, Pages 1-18; Zhang et al., 2020, “Potent human neutralizing antibodies elicited by SARS-CoV-2 infection”, bioRxiv, Mar. 26, 2020, Pages 1-42. Additional suitable antibodies can be made by persons of ordinary skill in the art using art known means.

In some embodiments, the antigen-binding domain is a nucleocapsid protein-binding antibody domain that binds an epitope of the nucleocapsid protein. In some embodiments, a nucleocapsid-binding antibody domain comprises a light chain sequence having at least 90% identity (e.g., at least 91%, at least 92%, at least 93%, at least 94%, at least 95%, at least 96%, at least 97%, at least 98%, or at least 99%) to the amino acid sequence of SEQ ID NO:20. In some embodiments, a nucleocapsid protein-binding antibody domain comprises a heavy chain sequence having at least 90% identity (e.g., at least 91%, at least 92%, at least 93%, at least 94%, at least 95%, at least 96%, at least 97%, at least 98%, or at least 99%) to the amino acid sequence of SEQ ID NO:21 or SEQ ID NO:64. In some embodiments, a nucleocapsid protein-binding antibody domain comprises an scFv sequence having at least 90% identity (e.g., at least 91%, at least 92%, at least 93%, at least 94%, at least 95%, at least 96%, at least 97%, at least 98%, or at least 99%) to the amino acid sequence of SEQ ID NO:22.

Exemplary nucleocapsid protein binding antibodies are identified in Table 2. For each antibody, the light chain and heavy chain sequences are identified together with the corresponding light chain and heavy chain CDR sequences. In some embodiments, a nucleocapsid protein-binding antibody domain comprises a light chain sequence having at least 90% identity (e.g., at least 91%, at least 92%, at least 93%, at least 94%, at least 95%, at least 96%, at least 97%, at least 98%, or at least 99%) to the amino acid sequence of a light chain sequence identified in Table 2. In some embodiments, a nucleocapsid protein-binding antibody domain comprises a heavy chain sequence having at least 90% identity (e.g., at least 91%, at least 92%, at least 93%, at least 94%, at least 95%, at least 96%, at least 97%, at least 98%, or at least 99%) to the amino acid sequence of a heavy chain sequence identified in Table 2. In some embodiments, a nucleocapsid protein-binding antibody domain comprises a light chain sequence having at least 90% identity (e.g., at least 91%, at least 92%, at least 93%, at least 94%, at least 95%, at least 96%, at least 97%, at least 98%, or at least 99%) to the amino acid sequence of a light chain sequence identified in one row in Table 2 and a heavy chain sequence having at least 90% identity (e.g., at least 91%, at least 92%, at least 93%, at least 94%, at least 95%, at least 96%, at least 97%, at least 98%, or at least 99%) to the amino acid sequence of a corresponding heavy chain sequence in the same row or a different row of Table 2. In some embodiments, a nucleocapsid protein-binding antibody domain comprises a light chain variable region comprising a CDRL1 sequence, a CDRL2 sequence, and a CDRL3 sequence from one row in Table 2; and a heavy chain variable region comprising a CDRH1 sequence, a CDRH2 sequence, and a CDRH3 sequence from the same row in Table 2. In some embodiments, a nucleocapsid protein-binding antibody domain comprises a light chain variable region comprising a CDRL1 sequence, a CDRL2 sequence, and a CDRL3 sequence from one row in Table 2; and a heavy chain variable region comprising a CDRH1 sequence, a CDRH2 sequence, and a CDRH3 sequence from a different row in Table 2. In some embodiments, a nucleocapsid protein-binding antibody domain comprises at least one of the CDR sequences set forth in SEQ ID NOs:15, 23-27, and 87-119. In some embodiments, a nucleocapsid protein-binding antibody domain comprises at least one of the CDRL sequences set forth in SEQ ID NOs: 23, 15, or 24, 87, 91, 95, 99, 103, 107, 110, and/or 114. In some embodiments, a nucleocapsid protein-binding antibody domain comprises at least one of the CDRH sequences set forth in SEQ ID NOs: 25-27, 88-90, 92-94, 96-98, 100-102, 104-106, 108, 109, 111-113, and/or 115-117.

TABLE 2

Nucleocapsid (N)-binding antibody sequences.

| | | | | | | | | |
|---|---|---|---|---|---|---|---|---|
| Antibody | Light chain | CDRL1 | CDRL2 | CDRL3 | Heavy chain | CDRH1 | CDRH2 | CDRH3 |
| N-binding clone H2 | SEQ ID NO:20 | SEQ ID NO:23 | SEQ ID NO:15 | SEQ ID NO:24 | SEQ ID NO:64 | SEQ ID NO:25 | SEQ ID NO:26 | SEQ ID NO:27 |
| N-binding clone 2 | SEQ ID NO:65 | SEQ ID NO:23 | SEQ ID NO:15 | SEQ ID NO:87 | SEQ ID NO:73 | SEQ ID NO:88 | SEQ ID NO:89 | SEQ ID NO:90 |
| N-binding clone 3 | SEQ ID NO:66 | SEQ ID NO:23 | SEQ ID NO:15 | SEQ ID NO:91 | SEQ ID NO:74 | SEQ ID NO:92 | SEQ ID NO:93 | SEQ ID NO:94 |
| N-binding clone 8 | SEQ ID NO:67 | SEQ ID NO:23 | SEQ ID NO:15 | SEQ ID NO:95 | SEQ ID NO:75 | SEQ ID NO:96 | SEQ ID NO:97 | SEQ ID NO:98 |
| N-binding clone 9 | SEQ ID NO:68 | SEQ ID NO:23 | SEQ ID NO:15 | SEQ ID NO:99 | SEQ ID NO:76 | SEQ ID NO: 100 | SEQ ID NO:101 | SEQ ID NO:102 |
| N-binding clone 12 | SEQ ID NO:69 | SEQ ID NO:23 | SEQ ID NO:15 | SEQ ID NO:103 | SEQ ID NO:77 | SEQ ID NO:104 | SEQ ID NO:105 | SEQ ID NO:106 |
| N-binding clone 14 | SEQ ID NO:70 | SEQ ID NO:23 | SEQ ID NO:15 | SEQ ID NO:107 | SEQ ID NO:78 | SEQ ID NO:25 | SEQ ID NO:108 | SEQ ID NO:109 |
| N-binding clone 15 | SEQ ID NO:71 | SEQ ID NO:23 | SEQ ID NO:15 | SEQ ID NO:110 | SEQ ID NO:79 | SEQ ID NO:111 | SEQ ID NO:112 | SEQ ID NO:113 |
| N-binding clone 24 | SEQ ID NO:72 | SEQ ID NO:23 | SEQ ID NO:15 | SEQ ID NO:114 | SEQ ID NO:80 | SEQ ID NO:115 | SEQ ID NO:116 | SEQ ID NO:117 |

Other nucleocapsid-binding antibodies are known. See, e.g., Terry et al., 2021, “Development of a SARS-CoV-2 nucleocapsid specific monoclonal antibody,” Virology 558:28-37 and Tian et al., 2021, “Epitope mapping of severe acute respiratory syndrome-related coronavirus nucleocapsid protein with a rabbit monoclonal antibody,” Virus Res. 300: 198445. Additional suitable antibodies can be made by persons of ordinary skill in the art using art known means.

The present disclosure provides protein biosensor V domains comprising sequences from antibodies that specifically or selectively bind a SARS-CoV-2 viral protein. As used herein, the terms specifically binds to, specific for, selectively binds and selective for a SARS CoV-2 viral protein mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

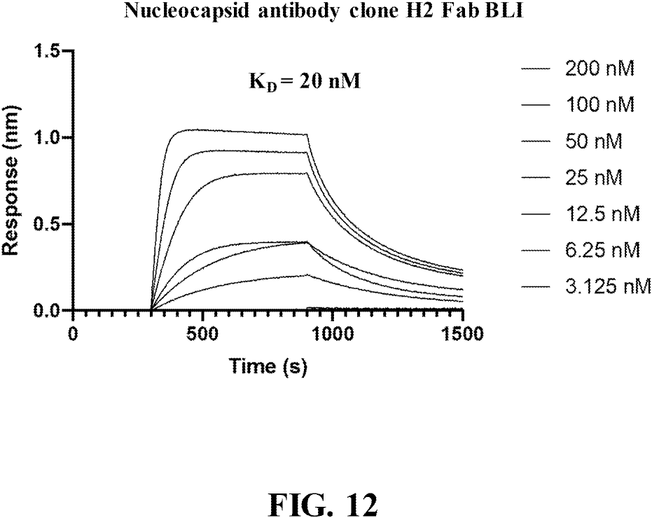

In some instances, binding affinity can be measured using titrations of purified target protein (e.g., spike or nucleocapsid protein) or a domain of the target protein (e.g., spike RBD) and yeast surface expressed antibodies, antibody fragments, or antibody fusion proteins as provided herein. Exemplary methods include those described above in the “ACE2 polypeptide V domain” section for ACE2 polypeptides and in Glasgow et al., 2020. In some instances, binding affinity can be measured by measuring the off rate of antibodies, antibody fragments, or antibody fusion proteins as provided herein bound to purified target protein (e.g., spike or nucleocapsid protein) or a domain of the target protein (e.g., spike RBD) in the presence of untagged inhibitor protein, as described for ACE2 polypeptides above, including the expression of binding affinity within a factor or 2 or within a factor of 3. Other methods of measuring binding affinity include ELISA, bio-layer interferometry, surface plasmon resonance, or kinetic exclusion assays (Kinexa®) as described in more detail above in the “ACE2 polypeptide V domain” section. In one embodiment, as described herein in Example 9 and shown in FIG. 12, an H2 Fab antibody fragment (clone H2, see Table 2) may have a K_{D} (binding affinity) for nucleocapsid protein of 20 nM when measured by BLI (see Example 1 for method). Titrations of purified and yeast surface expressed proteins can be used to determine the actual K_{D} and apparent K_{D}, respectively. The K_{D} range in which measurements are accurate for different analytical methods may vary. One of skill in the art will appreciate that, within the accurate range, these methods will result in similar binding affinity measurements or similar trends in relative binding affinities for the various antibodies, antibody fragments, and antibody fusion proteins described herein.

As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. As used herein, the term antibody also encompasses an antibody fragment, for example, an antigen binding fragment. Antigen binding fragments comprise at least one antigen binding domain. One example of an antigen binding domain is an antigen binding domain formed by a VH-VL dimer. Antibodies and antigen binding fragments can be described by the antigen to which they specifically bind.

The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity. Each VH and VL generally comprises three CDRs and four FRs, arranged in the following order (from N-terminus to C-terminus): FR1 - CDR1 - FR2 - CDR2 - FR3 - CDR3 - FR4. The CDRs are involved in antigen binding, and confer antigen specificity and binding affinity to the antibody. (See Kabat et al., (1991) Sequences of Proteins of Immunological Interest 5th ed., Public Health Service, National Institutes of Health, Bethesda, MD.) CDR sequences on the heavy chain (VH) may be designated as CDRH1, 2, 3, while CDR sequences on the light chain (VL) may be designated as CDRL1, 2, 3.

### B. Detection Moieties

As discussed above, the protein biosensors provided herein comprise pairs of fusion proteins, wherein each fusion protein of the pair comprises a viral protein-binding domain and a detection moiety. In some embodiments, the detection moiety is a polypeptide domain (i.e., a detection moiety domain or a D domain). In some embodiments, the detection moiety domains of a protein biosensor comprise split reporters comprising complementary fragments. In some embodiments, the detection moiety is a nucleic acid (e.g., an oligonucleotide) or another detectable moiety (e.g., a chemical functional group, a fluorophore, biotin). In some embodiments, the detection moieties of a protein biosensor associate when bound in proximity to each other (optionally in the presence of accessory reagents).

### Protein Detection Moieties

In some embodiments, polypeptide detection moiety domains comprise split reporter protein fragments. In some embodiments, each of the complementary fragments of the split reporter protein is individually inactive and, when all the complementary fragments associate with one another, they may form an active (e.g., enzymatically active) protein complex, which can be detected. Each of the complementary fragments of a “split reporter protein” can be referred to as a “polypeptide fragment”, or a “peptide fragment,” e.g., a first peptide fragment and a second peptide fragment. In some embodiments, the fragments of the split reporter proteins have low affinity for each other and must be brought together by other interacting proteins fused to them. The ability to turn on the split reporter protein activity can be exploited to monitor protein interactions by fusing each peptide fragment of the split protein to different proteins that have affinity for one another, or, as demonstrated herein, by fusing each peptide fragment of the split protein to different proteins that are able to bind to the same viral protein or to two viral proteins in close proximity. In some embodiments, the interaction between these different proteins creates a high local concentration of the peptide fragments, thereby causing the separate fragments of the split protein to bind to one another to form an active protein complex.

In some embodiments, the split reporter is a split-luciferase. In some embodiments, the luciferase is a split-nanoluciferase. Split-nanoluciferases are commercially available, for example, NanoBiT® from Promega (Madison, WI). The NanoBiT system comprises two subunits: Small BiT (SmBiT), an 11 amino acid peptide (SEQ ID NO:28), and Large Bit (LgBiT), a 17.6 kDa subunit (SEQ ID NO:29) that binds weakly to SmBiT (K_{D} = 190 µM). When the SmBiT and LgBiT domains are in close proximity, the two subunits come together to form an active luciferase. See U.S. Pat. No. 9,797,889 B2 and Dixon et al., 2016, “NanoLuc Complementation Reporter Optimized for Accurate Measurement of Protein” ACS Chem. Biol. 11:400-408, both incorporated herein by reference.

In some embodiments, the first fusion protein of the sensor comprises a first peptide fragment having greater than 40% sequence identity with SEQ ID NO:29 (e.g., >40%, >45%, >50%, >55%, >60%, >65%, >70%, >75%, >80%, >85%, >90%, >95%, >98%, >99%), and/or the second fusion protein of the sensor comprises a second peptide fragment comprising SEQ ID NO: 28, and a detectable bioluminescent signal is produced or substantially increased when the first peptide fragment contacts the second peptide fragment. In some embodiments, the second peptide fragment has a sequence having one, two, or three single amino acid mutations (substitutions, deletions, or insertions) relative to SEQ ID NO:28.

In one example, the protein biosensor detection moiety domains comprise split fragments of a luciferase (e.g., a first peptide fragment and a second peptide fragment). For example, the protein biosensor detection moiety domains may comprise SmBiT and LgBiT, split fragments of a Nanoluciferase (NanoLuc®) of the NanoBiT® system (Promega), as SARS-CoV-2 virus sensors.

Other luciferase-based split reporter systems may be used in the present invention. See, Cassonnet et al., 2011, “Benchmarking a luciferase complementation assay for detecting protein complexes” Nature Methods. 8 (12): 990-992. For example, other systems include ReBiL (Li et al.. 2014, “A versatile platform to analyze low-affinity and transient protein-protein interactions in living cells in real time” Cell Reports 9 (5): 1946-58) and gaussia princeps luciferase (GLuc) (Neveu et al., 2012, “Comparative analysis of virus-host interactomes with a mammalian high-throughput protein complementation assay based on Gaussiaprinceps luciferase” Methods 58 (4): 349-359).

Additional reporter proteins include horseradish peroxidase or HRP (Martell at al. (2016). “A split horseradish peroxidase for the detection of intercellular protein-protein interactions and sensitive visualization of synapses”. Nature Biotechnology. 34 (7): 774-80), engineered soybean ascorbate peroxidase (APEX2); β-lactamase (Park et al. (2007). “Bacterial beta-lactamase fragmentation complementation strategy can be used as a method for identifying interacting protein pairs,” Journal of Microbiology and Biotechnology. 17 (10): 1607-15), β-galactosidase (Rossi et al. (1997). “Monitoring protein-protein interactions in intact eukaryotic cells by beta-galactosidase complementation,” Proc. National Acad. Sci. USA 94 (16): 8405-10), dihydrofolate reductase (Tarassov et al. (2008). “An in vivo map of the yeast protein interactome,” Science 320 (5882): 1465-70), Green Fluorescent Protein (GFP) and GFP variants (Barnard et al. (2010). “Split-EGFP Screens for the Detection and Localisation of Protein-Protein Interactions in Living Yeast Cells,” Methods in Molecular Biology 638: 303-17; Blakeley et al. (2012). “Split-superpositive GFP reassembly is a fast, efficient, and robust method for detecting protein-protein interactions in vivo,” Molecular BioSystems. 8 (8): 2036-40; Cabantous et al. (2013). “A new protein-protein interaction sensor based on tripartite split-GFP association,” Scientific Reports,. 3: 2854; MacDonald et al. (2006). Nat Chem Biol 2006, 2, 329-337; Hu et al. (2003) Nat Biotechnol 2003, 27, 539-45), ubiquitin (Duenkler et al. (2012). “Detecting Protein-Protein Interactions with the Split-Ubiquitin Sensor,” Methods in Molecular Biology 786: 115-30), Tobacco Etch Virus (TEV) protease (Wehr et al. (2006) “Monitoring regulated protein-protein interactions using split TEV,” Nature Methods 3 (12): 985-93), focal adhesion kinase (Ma et al. (2014) “A new protein-protein interaction sensor based on tripartite split-GFP association,” Scientific Reports 3: 2854), and infrared fluorescent protein IFP1.4 (Tchekanda et al. (2014) “An infrared reporter to detect spatiotemporal dynamics of protein-protein interactions,” Nature Methods 11 (6): 641-4); Michnick et al., Nat Rev Drug Discov 6, 569-82 (2007); Remy & Michnick, Methods Mol Biol 1278, 467-81 (2015); Morrell et al., FEBS Lett 583, 1684-91 (2009)). Additional reporter proteins also include split protein complementation (Shekhawat & Ghosh, Curr Opin Chem Biol 15: 789-97 (2011)) and bimolecular fluorescence complementation (Miller et al., 2015, J Mol Biol 427: 2039-55; Kerppola, T. K., 2009, Chem Soc Rev 38: 2876-2886). See also Shekhawat and Ghosh, 2011, “Split-protein systems: beyond binary protein-protein interactions,” -Curr. Opin. Chem. Biol. 15 (6): 789-797.

### Non-Protein Detection Moieties

The reporter moieties (also referred to as detection moieties in this disclosure) of the split reporter can also be nucleic acids or other moieties (e.g., chemical functional groups, fluorophores, biotin) that associate when bound in proximity to each other (optionally in the presence of accessory reagents).

In some embodiments, proximity extension assays and/or proximity ligation assays are used to detect SARS-CoV-2 virus. In proximity extension assays and proximity ligation assays, oligonucleotide probes (i.e. a pair of nucleic acid moieties), each attached to a viral protein-binding domain in a fusion protein as described herein, are brought into proximity in the presence of a virus to which the viral protein-binding domains bind. If the fusion proteins bind close together (e.g., the viral protein-binding domains bind the same spike protein or bind two different viral proteins on the same virion), the nucleic acid moieties interact by hybridization to each other, or hybridization to a common splint oligonucleotide, to form a complex. The complex can then be detected by ligation, extension and/or amplification of the nucleic acid complex. See, e.g., U.S. Pat. No. 6,878,515; U.S. Pat. No. 7,306,904; Fredriksson et al., 2002, “Protein detection using proximity-dependent DNA ligation assays.” Nat. Biotechnol. 20:473-477; Lundberg et al., 2011, “Homogeneous antibody-based proximity extension assays provide sensitive and specific detection of low-abundant proteins in human blood” Nucleic Acids Res. 39:e102. In these approaches, one fusion protein comprises the first viral protein-binding domain linked to the first nucleic acid probe and the other fusion protein comprises the second viral protein-binding domain linked to the second nucleic acid probe, and they are used in a system that is adapted for a proximity ligation assay, proximity extension assay or other nucleic acid based proximity assay (e.g., the two oligonucleotides are partially complementary to each other or are both partially complementary to an oligonucleotide in the mixture).

In some embodiments, the reporter moieties can be moieties that are not nucleic acids or proteins. In some embodiments, the moieties may comprise fluorophores, and the association of the reporter moieties may be detected using, e.g., fluorescence resonance energy transfer.

### C. Linker Domains

The fusion proteins of the protein biosensors described herein may include linker domains. In some embodiments, the fusion protein domains (e.g., a V domain and a detection moiety domain) are joined or connected via a linker, e.g., a peptide linker. In some embodiments, a viral protein-binding domain is fused to one member of the complementary portions of a split reporter (e.g., a peptide fragment or an oligonucleotide) via a linker. In some embodiments, one member of the two complementary portions of the split reporter is fused to a first viral protein-binding domain via a first linker, and the second viral protein-binding domain is fused to the other member of the two complementary portions of the split reporter via a second linker. The first linker and the second linker may have the same amino acid sequence or different amino acid sequences. The first linker and the second linker may also be of the same or different length.

A linker sequence may increase the range of orientations that may be adopted by the domains of the fusion protein. A linker sequence may be optimized to produce desired effects in the fusion protein. Aspects of linker design and considerations are described, for example, in Koerber et al., 2015, “An improved single-chain Fab platform for efficient display and recombinant expression,” J Mol Biol 427(2):576-586, Chen, X. et al., Adv Drug Deliv Rev. 2013 Oct 15; 65(10): 1357-1369, and Klein, J.S. et al. 2014 Protein Eng. Des. Sel. 27(10):325-330.

A peptide linker may be, for example, 5 to 60 or more amino acids in length (e.g., 5 aa, 10 aa, 15 aa, 25 aa, 35 aa, 40 aa, 45 aa, 50 aa, 55 aa, or 60 aa). In some embodiments, the length of a linker may affect the sensitivity of virus detection when using protein biosensors comprising the linker. Depending on length, linker sequence may have various conformations in secondary structure, such as helical, β-strand, coil/bend, and turns. In some instances, a linker sequence may have an extended conformation and function as an independent domain that does not interact with the adjacent protein domains. Linker sequences may be flexible or rigid. Flexible linkers provide a certain degree of movement or interaction between the polypeptide domains and are generally rich in small or polar amino acids such as Gly and Ser (e.g., at least 90%, at least 95%, at least 98%, at least 99%, or all of the amino acid residues of the linker are either Gly or Ser). A rigid linker can be used to keep a fixed distance between the domains and to help maintain their independent functions.

In some embodiments, the linker comprises SEQ ID NO:30 (GSSGGGGSGGGGSGGGGSGGGG). In some embodiments, the linker comprises SEQ ID NO:31 (GSSGGGGSGGGGSGGGG). In some embodiments, the linker comprises SEQ ID NO:32 (GSSGGGGSGGGG). In some embodiments, the linker comprises SEQ ID NO:33 (CSGGGGSGGGG). In some embodiments, the linker comprises SEQ ID NO:54 (TSSGGGGENLYFQSSGGGSGGG). In some embodiments, the linker comprises SEQ ID NO:118 (GGSGSAGG). In some embodiments, the linker comprises SEQ ID NO:119 (GGSGSGGGGS). In some embodiments, the linker comprises SEQ ID NO:120 (GGGSG). In some embodiments, the linker comprises one or more repeats of GGGGS (SEQ ID NO:34) and/or one or more repeats of GSSGSS (SEQ ID NO:35). Additional exemplary peptide linkers include, but are not limited to, peptide linkers comprising SEQ ID NO:36 (SGSETPGTSESATPE), SEQ ID NO:37 (SGSETPGTSESATPES), SEQ ID NO:38 ((GGGGS)₃), SEQ ID NO:39 ((GGGGS)s), SEQ ID NO:40 ((GGGGS)₁₀), GGGGGGGG (SEQ ID NO:41) and GSAGSAAGSGEF (SEQ ID NO:42), SEQ ID NO:43 (A(EAAAK)₃A), or SEQ ID NO:44 (A(EAAAK)₁₀A). Additional non-limiting exemplary linkers that can be used include those disclosed in Chen et al., Adv. Drug. Deliv. Rev. 65 (10): 1357-1369 (2014) and Rosemalen et al., Biochemistry 2017, 56, 50, 6565-6574, the entire contents of both of which are herein incorporated by reference.

The fusion proteins may also comprise spacer sequences within or between domains. In some embodiments, spacer sequences may increase the range of orientations that may be adopted by the domains of a fusion protein described herein. In some embodiments, the split reporter protein fragment is separated from another domain (e.g., Fc domain, ACE2 domain) by a spacer sequence. Spacers may be, for example, 2 to 35 or more amino acids in length (e.g., 2 aa, 4 aa, 5 aa, 10 aa, 15 aa, 25 aa or 35 aa).

### D. Dimerization Domains

In some embodiments, the fusion proteins described in this disclosure comprise a dimerization domain. In some embodiments, fusion proteins comprising dimerization domains are able to associate with one another to form a dimer. As demonstrated in the Examples herein and in Glasgow et al., 2020, fusion protein dimerization can increase binding affinity of the fusion protein for a target viral protein. For example, ACE2 fusion proteins that can form a dimer bind spike RBD with higher affinity than ACE2 fusion proteins that cannot forma dimer. In some embodiments, the dimerization domain is an ACE2 collectrin domain, as described above.

In some embodiments, the dimerization domain is an Fc domain. The Fc domain is able to form a homodimer. In some embodiments, the dimerization domain is an Fc domain and a hinge domain. Any desired hinge and/or Fc domain may be used. In some embodiments, a human Fc sequence or human Fc and hinge sequence is used. In some embodiments, an Fc sequence or Fc and hinge sequences from a nonhuman primate is used. In some embodiments, an Fc sequence is used with a heterologous hinge sequence (e.g., a human Fc sequence with a nonhuman primate hinge sequence or a nonhuman primate Fc sequence with a human hinge sequence). In some embodiments, the Fc domain is a human IgG1 Fc domain with the amino acid sequence set forth in SEQ ID NO:45, alone or together with a human IgG1 hinge domain (SEQ ID NO:46). The amino acid sequence of a human IgG1 Fc domain together with a human IgG1 hinge domain is set forth in SEQ ID NO:47. In some embodiments, the dimerization domain comprises SEQ ID NO:47. In some embodiments, the dimerization domain comprises SEQ ID NO:48.

In some embodiments, a dimerization domain other than an Fc domain is used. There are a wide array of protein dimerization domains known in the art, including commercially available constructs that can be used to express fusion proteins (e.g., iDimerize® system, Takara Bio USA).

### E. Additional Domains

The fusion proteins of the protein biosensors described herein may include additional domains in addition to viral protein-binding domains, detection moieties, and optional linker domains.

Any of the polypeptides or proteins described herein can further comprise a detectable moiety, for example, a fluorescent protein or fragment thereof. Examples of fluorescent proteins include, but are not limited to, yellow fluorescent protein (YFP, for example, Venus), green fluorescent protein (GFP), and red fluorescent protein (RFP) as well as derivatives, for example, mutant derivatives, of these proteins. See, for example, Chudakov et al., “Fluorescent Proteins and Their Applications in Imaging Living Cells and Tissues,” Physiological Reviews 90(3): 1103-1163 (2010); and Specht et al., “A Critical and Comparative Review of Fluorescent Tools for Live-Cell Imaging,” Annual Review of Physiology 79: 93-117 (2017).

Any of the polypeptides or proteins described herein can further comprise a domain or sequence useful for protein isolation. In some embodiments, the polypeptides comprise an affinity tag, for example an AviTag™ (SEQ ID NO:49), a Myc tag (SEQ ID NO:50), a polyhistidine tag (e.g., 8XHis tag (SEQ ID NO:51)), an albumin-binding protein, an alkaline phosphatase, an AU1 epitope, an AU5 epitope, a biotin-carboxy carrier protein (BCCP), or a FLAG epitope, to name a few. In some embodiments, the affinity tags are useful for protein isolation. See, Kimple et al., “Overview of Affinity Tags for Protein Purification,” Curr. Protoc. Protein Sci. 73: Unit-9.9 (2013). In some embodiments, the polypeptides or proteins described herein comprise a signal sequence useful for protein isolation, for example a mutated Interleukin-2 signal peptide sequence (SEQ ID NO:52), which promotes secretion and facilitates protein isolation. See, for example, Low et al., “Optimisation of signal peptide for recombinant protein secretion in bacterial hosts,” Applied Microbiology and Biotechnology 97:3811-3826 (2013). In some embodiments, the polypeptides or proteins described herein comprise a protease recognition site, for example, TEV protease cut site (SEQ ID NO:53).Such protease recognition sites may be useful for, among other things, allowing removal of a signal peptide or affinity purification tag following protein isolation. In some embodiments, a TEV protease cut site is part of a linker domain (e.g., SEQ ID NO:54).

### F. Protein Modifications

In some embodiments, the fusion proteins provided herein comprise amino acid substitutions that improve binding or other properties. For example, one or more cysteine substitutions, or substitutions with noncanonical amino acids containing long side-chain thiols, may be introduced into the polypeptides that can form disulfide bonds between two polypeptides that have interacted to form a dimer. In some embodiments, the substitutions improve polypeptide stability. For example, the ACE2 polypeptides described above may comprise substitution of the tryptophan residue at position 69 (with reference to SEQ ID NO: 1) with a valine residue, a lysine residue, or an isoleucine residue.

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al., “Protein engineering with unnatural amino acids,” Curr. Opin. Struct. Biol. 23(4): 581-587 (2013); Xie et la. “Adding amino acids to the genetic repertoire,” 9(6): 548-54 (2005)); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

In any of the polypeptides described herein, where a specific amino acid sequence is recited, embodiments comprising a sequence having at least 90% (e.g. 90%, 91%, 92%, 93%, 94%, 95%, 96%, 97%, 98%, or 99%) identity to the recited sequence are also provided.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=-2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat′l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

Sequence identity can be also be determined by inspection. For example, the sequence identity between sequence A and sequence B, aligned using the software above or manually (to maximize alignment), can be determined by dividing the length of sequence A, minus the number of gap residues in sequence A, minus the number of gap residues in sequence B, by the sum of the residue matches between sequence A and sequence B, times one hundred.

### V. Nucleic Acids, Constructs, Vectors, and Host Cells

Recombinant nucleic acids encoding any of the polypeptides or proteins described herein are also provided. As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

Also provided is a DNA construct comprising a promoter operably linked to a recombinant nucleic acid described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of the modified polypeptide. The cassette may additionally contain at least one additional gene or genetic element to be cotransformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette will include in the 5′ to 3′ direction of transcription: a transcriptional and translational initiation region (i.e., a promoter), a polynucleotide disclosed herein, and a transcriptional and translational termination region (i.e., termination region) functional in the cell or organism of interest. The promoters described herein are capable of directing or driving expression of a coding sequence in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.); Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid gene (See generally, Sambrook et al. Molecular Cloning: A Laboratory Manual, 4^{th} ed., Cold Spring Harbor Laboratory Press, Cold Spring Harbor, 2012). The vector, for example, can be a plasmid.

There are numerous E. coli expression vectors known to one of ordinary skill in the art, which are useful for the expression of a nucleic acid. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide of the present invention, wherein the nucleic acid can be expressed by a yeast cell. More specifically, the nucleic acid can be expressed by Pichia pastoris or S. cerevisiae.

Various commericial vectors for the expression of antibodies, antigen-binding fragments thereof, and fusion proteins are available. For example, the pFUSE vectors available from Invitrogen offer a variety of antibody fusion protein formats (e.g., IgG and scFv formats). Methods for the expression of Fab fragments are also described, e.g., in Hornsby et al., 2015, “A high through-put platform for recombinant antibodies to folded proteins,” Mol. Cell Proteomics 14(10):2833-2847. In some embodiments, antibodies or antibody fragments (e.g., Fab fragments) include a heavy chain and a light chain sequence, which may be expressed from the same vector or different vectors. In some embodiments, the heavy chain and light chain sequences are able to interact to form an antibody or antibody fragment. In some embodiments, the heavy chain and light chain sequences are expressed as a single fusion polypeptide sequence (e.g., scFv).

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, genticin or G418 resistance, or other genes or phenotypes suitable for use as selectable markers, or methotrexate resistance for gene amplification. A number of suitable host cell lines capable of secreting intact human proteins have been developed in the art, and include CHO cells, HeLa cells, COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include the nucleic acids as described herein under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Insect cells also permit the expression of the polypeptides. Recombinant proteins produced in insect cells with baculovirus vectors undergo post-translational modifications similar to that of wild-type mammalian proteins.

A host cell comprising a nucleic acid, a DNA construct, or a vector described herein is also provided. The host cell can be an in vitro, ex vivo, or in vivo host cell. Populations of any of the host cells described herein are also provided. A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook (supra); Ausubel et al. (eds.), 1999, “Short protocols in molecular biology, 4^{th} edn.,” New York, NY: Wiley; and Berger et al. (eds.), 1996, “Methods in Enzymology, a Guide to Molecular Cloning Techniques, Vol. 152,” San Diego, CA: Academic Press; as well as Freshney (1994) Culture of Animal Cells, a Manual of Basic Technique, 3^{rd} Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4^{th} Ed. W.H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be an HEK293T cell, a Chinese hamster ovary (CHO) cell, a COS-7 cell, a HELA cell, an avian cell, a myeloma cell, a Pichia cell, an insect cell, or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host.

As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid, for example, a nucleic acid encoding a recombinant protein described herein, into a host cell.

Any of the fusion proteins or polypeptides described herein can be purified or isolated from a host cell or population of host cells. For example, a recombinant nucleic acid encoding any of the proteins described herein can be introduced into a host cell under conditions that allow expression of the protein. In some embodiments, the recombinant nucleic acid is codon-optimized for expression. After expression in the host cell, the recombinant protein can be isolated or purified using purification methods known in the art. In some embodiments, a recombinant nucleic acid encoding a recombinant ACE2 polypeptide fusion protein can be introduced into a host cell under conditions that allow expression thereof, with the expressed polypeptide forming a protein dimer. In some embodiments, a recombinant nucleic acid encoding a fusion protein comprising a recombinant ACE2 polypeptide and a dimerization domain can be introduced into a host cell under conditions that allow expression of the fusion protein, with the expressed polypeptide forming a protein dimer. After expression in the host cell, the protein dimer can be isolated or purified using purification methods known in the art. In some embodiments, the fusion protein is isolated as a monomer and allowed to dimerize in vitro.

Following expression, the fusion proteins or polypeptides can be isolated. Proteins can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

In vitro methods are also suitable for preparing fusion proteins or polypeptides. For example, digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen. The Fab fragments produced in antibody digestion can also contain the constant domains of the light chain and the first constant domain of the heavy chain. Fab’ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing fusion proteins is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). A protein provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form a fusion protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J.Biol.Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Methods for determining the yield or purity of a purified protein are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

An “isolated” or “purified” polypeptide or protein is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, 1%, 0.5%, or 0.1% (total protein) of contaminating protein. When the protein of the invention or its biologically active portion is recombinantly produced, optimally culture medium represents less than about 30%, 20%, 10%, 5%, 1%, 0.5%, or 0.1% (by concentration) of chemical precursors or non-protein-of-interest chemicals.

### VI. Exemplary Protein Biosensors

The protein biosensors provided herein may take different forms. A protein biosensor typically includes two fusion proteins, each comprising a split reporter fragment and a viral protein-binding domain. In some embodiments, the split reporter fragment is fused to the end (i.e., the C terminus or the N terminus) of a viral protein-binding domain (e.g., the ACE2 polypeptide domain, the spike-binding antibody domain, or nucleocapsid binding antibody domain) in a fusion protein of a protein biosensor. In some embodiments, the split reporter fragment is internal (i.e., flanked by other fusion protein domains). In some embodiments, the protein biosensor comprises at least two fusion proteins, in which each of the split reporter fragments is fused to the N terminus of a viral protein-binding domain. In some embodiments, the protein biosensor comprises two fusion proteins, in which each of the split reporter fragments is fused either to the C terminus or to the N terminus of a viral protein-binding domain. In some embodiments, the protein biosensor comprises two fusion proteins; in one fusion protein, the split reporter fragment is fused to the C terminus of a viral protein-binding domain, and in the other fusion protein, the split reporter fragment is fused to the N terminus of a viral protein-binding domain.

In some embodiments, the two viral protein-binding domains in the protein biosensor are the same, i.e., having the same sequence. In some cases, the two viral protein-binding domains in the protein biosensor are different, i.e., having different sequences. In some embodiments, the protein biosensor is capable of detecting the presence of virus particles as long as the viral protein-binding domains can both bind to the same viral protein or to two different viral proteins in close proximity.

As described above, the fusion proteins described herein may comprise one or more additional domains or sequences (e.g., dimerization domains, linkers, spacers, affinity tags, etc.). It will be understood that the one or more additional domains in a fusion protein may be arranged in any order, relative to each other, the viral protein-binding domain, and/or the detection moiety. In some embodiments, the arrangement of the additional domains is selected based on the desired properties of the fusion protein. For example, fusion protein may comprise a spacer between a dimerization domain and a viral protein-binding domain to promote increased binding affinity. As another example, a fusion protein may comprise an affinity tag at the C terminus or N terminus to facilitate protein purification.

In some embodiments, the same one or more additional domains may be present in both fusion proteins of a protein biosensor. For example, both fusion proteins may comprise the same linker domain. In some embodiments, different one or more additional domains may be present in both fusion proteins of a protein biosensor. For example, one fusion protein of a protein biosensor may comprise a dimerization domain, and the other fusion protein of the protein biosensor may comprise a linker domain. In some embodiments, only one of the fusion proteins of a protein biosensor may comprise the one or more additional domains.

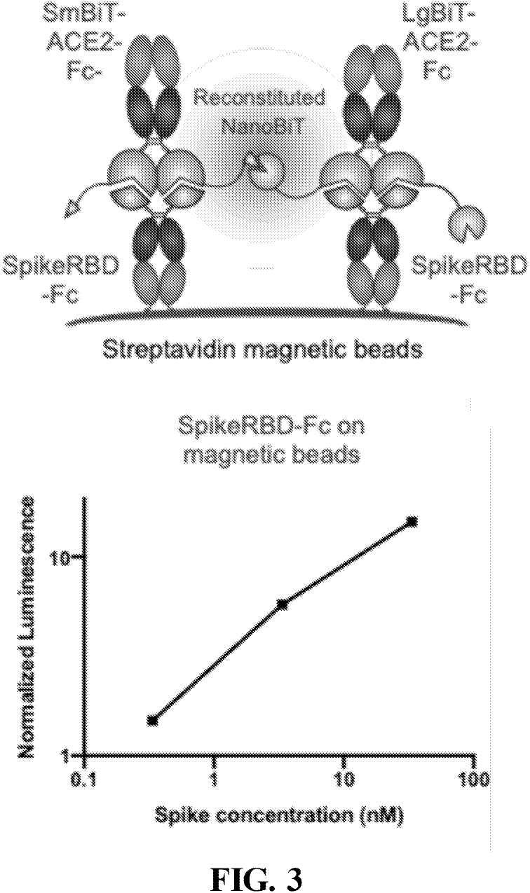

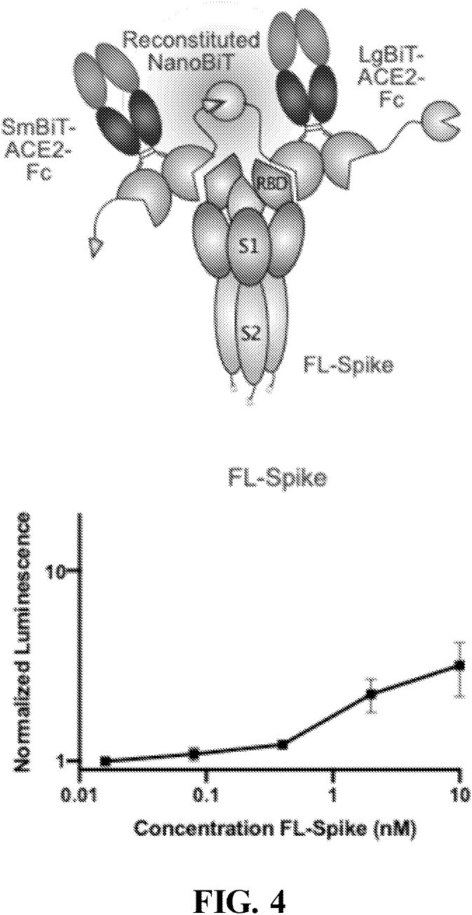

As described in the Examples, ACE2-Fc binds Spike with inter-Spike avidity but limited intra-Spike avidity (e.g., Examples 1-5 and FIG. 4). This finding has significant implications on the design strategy of a split-reporter based viral detection assay. For detecting two Spike molecules on the viral surface, two RBD-binding domains (e.g. a protein biosensor with two viral protein-binding domains comprising an ACE2 polypeptide can be used to enable reconstitution of the split reporters as described in Examples 4-6 and FIGS. 3-8. However, for detecting single Spike protein, it may be preferable to use only one ACE2 or ACE2-competitive binder, because a Spike trimer generally only presents one of the three RBD domains for binding to ACE2. Thus, in some embodiments, where detection of a single Spike protein may be desired, the first and second viral protein-binding domains may be selected to bind to different sequences in the Spike protein.

The protein biosensors provided herein comprise a pair of fusion proteins that each bind to a viral protein (the “protein target” of the fusion protein). The protein biosensors may comprise fusion proteins with any combination of protein targets (e.g., spike protein, nucleocapsid protein). In some embodiments, both fusion proteins of a protein biosensor target (bind to) the spike protein. In some embodiments, one fusion protein of a protein biosensor targets the spike protein through binding to an RBD domain, and the other fusion protein of the protein biosensor targets the spike protein through binding to another part (region) of the spike protein or through noncompetitive binding to the same RBD domain. In some embodiments, both fusion proteins of a protein biosensor target nucleocapsid protein. In some embodiments, one fusion protein of a protein biosensor targets the spike protein, and the other fusion protein of the protein biosensor targets the nucleocapsid protein. It will be recognized that any of the fusion proteins described herein above can be selected for use in a protein biosensor based on the desired protein target or targets. For example, a fusion protein comprising an ACE2 polypeptide may be selected for binding to the spike protein as the protein target, while a fusion protein comprising a nucleocapsid-binding antibody may be selected for binding to the nucleocapsid protein as the protein target.

In one aspect, provided herein are fusion proteins that comprise 1) an RBD-binding ACE2 polypeptide domain and a peptide fragment of a split reporter protein or a reporter moiety; 2) a spike-binding antibody domain and a peptide fragment of a split reporter protein or a reporter moiety; or 3) a nucleocapsid-binding antibody domain and a peptide fragment of a split reporter protein or a reporter moiety. Also provided are compositions comprising two of the fusion proteins, wherein the split reporter proteins of the fusion proteins are complementary fragments of a reporter protein. Also provided are compositions comprising two of the fusion proteins, wherein the reporter moieties of the fusion proteins are oligonucleotides that are partially complementary to each other or are both partially complementary to an additional oligonucleotide (e.g., a splint oligonucleotide, as described above).

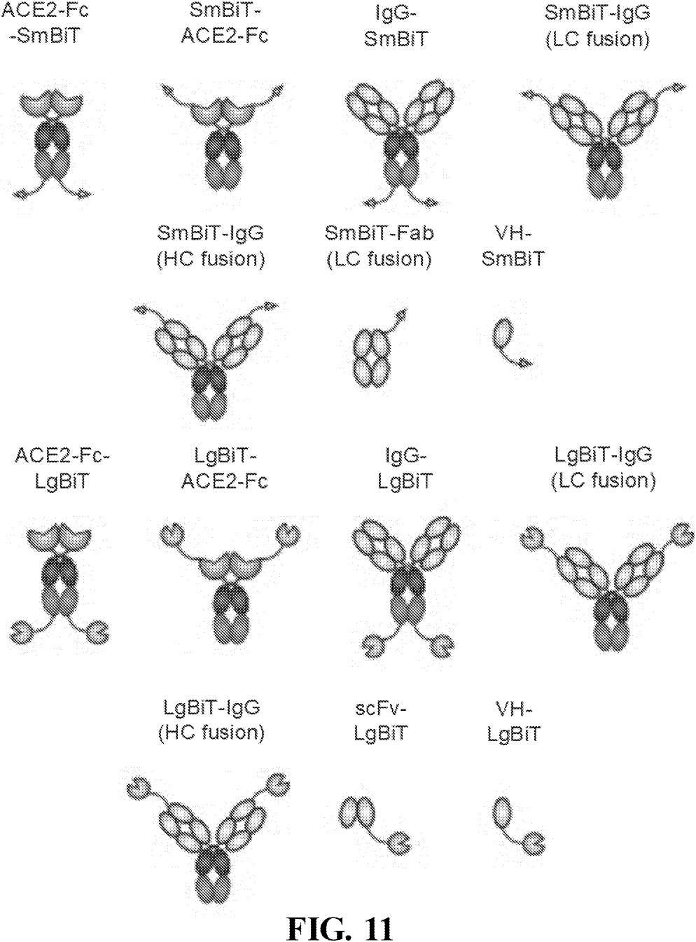

Provided below are several exemplary protein biosensor fusion proteins for targeting spike or nucleocapsid proteins. Schematic depictions of exemplary protein biosensor fusion proteins are also provided in FIG. 11. These examples are not intended to be limiting. It will also be appreciated that any of the variations, modifications, and/or additional domains described above may be added to these example fusion proteins within the scope of this disclosure. For example, an exemplary fusion protein with a wild-type ACE2 polypeptide sequence may be modified with any of the amino acid substitutions described above. In the following sections, construct domains are listed in order, beginning at the amino-terminus of the fusion protein and progressing to the carboxy-terminus.

### A. ACE2 Fusion Proteins

In some embodiments, the protein biosensors comprise at least one fusion protein comprising an ACE2 polypeptide domain and a split reporter protein fragment domain (e.g., SmBiT, LgBiT). In some embodiments, the fusion protein comprises an Fc domain (e.g., dimeric human IgGl Fc) and/or linker and hinge (e.g., human IgGl hinge) domains to allow flexible positioning of the two ACE2 domains in dimeric constructs.

Exemplary fusion proteins comprising an ACE2 polypeptide domain and a split reporter fragment include: ACE2-Fc-SmBiT; ACE2-Fc-LgBiT; SmBiT-ACE2-Fc; and LgBiT-ACE2-Fc (FIG. 11). In some embodiments, the fusion proteins are used in a viral detection assay as monomers. In some embodiments the fusion proteins are used in a viral detection assay as dimers. Exemplary fusion protein sequences are provided in Table 3, where full sequences are shown in the “Fusion protein” column, and domains in order from the amino-terminus to the carboxy-terminus are shown in the “Domains” column.

TABLE 3

Exemplary ACE2 fusion protein sequences.

| | |
|---|---|
| Fusion protein | Domains |
| ACE2-Fc-5aa-SmBiT (SEQ ID NO:55) | ACE2 signal sequence and peptidase domain (SEQ ID NO:5); linker with TEV protease cut site (SEQ ID NO:54); human IgGl hinge and Fc domain (SEQ ID NO:47); linker (1 repeat of SEQ ID NO:34); SmBit (SEQ ID NO:28) |
| ACE2-Fc-5aa-LgBiT (SEQ ID NO:56) | ACE2 signal sequence and peptidase domain (SEQ ID NO:5); linker with TEV protease cut site (SEQ ID NO:54); human IgGl hinge and Fc domain (SEQ ID NO:47); linker (1 repeat of SEQ ID NO:34); LgBit (SEQ ID NO:29) |
| ACE2-Fc-15aa-LgBiT (SEQ ID NO:57) | ACE2 signal sequence and peptidase domain (SEQ ID NO:5); linker with TEV protease cut site (SEQ ID NO:54); human IgGl hinge and Fc domain (SEQ ID NO:47); linker (3 repeats of SEQ ID NO:34); LgBit (SEQ ID NO:29) |
| ACE2-Fc-25aa-LgBiT (SEQ ID NO:58) | ACE2 signal sequence and peptidase domain (SEQ ID NO:5); linker with TEV protease cut site (SEQ ID NO:54); human IgGl hinge and Fc domain (SEQ ID NO:47); linker (5 repeats of SEQ ID NO:34); LgBit (SEQ ID NO:29) |
| ACE2-Fc-35aa-LgBiT (SEQ ID NO:59) | ACE2 signal sequence and peptidase domain (SEQ ID NO:5); linker with TEV protease cut site (SEQ ID NO:54); human IgGl hinge and Fc domain (SEQ ID NO:47); linker (7 repeats of SEQ ID NO:34); LgBit (SEQ ID NO:29) |
| SmBiT-10aa-ACE2-Fc (SEQ ID NO:60) | SmBit (SEQ ID NO:28); linker (2 repeats of SEQ ID NO:34); ACE2 ectodomain peptidase domain; linker with TEV protease cut site (SEQ ID NO:54); human IgGl hinge and Fc domain (SEQ ID NO:47); linker (1 repeat of SEQ ID NO:34); AviTag (SEQ ID NO:51) |
| LgBiT-10aa-ACE2-Fc (SEQ ID NO:61) | LgBit (SEQ ID NO:29); linker (2 repeats of SEQ ID NO:34); ACE2 ectodomain peptidase domain; linker with TEV protease cut site (SEQ ID NO:54); human IgGl hinge and Fc domain (SEQ ID NO:47); linker (1 repeat of SEQ ID NO:34); AviTag (SEQ ID NO:51) |

### B. Antibody Fusion Proteins

In some embodiments, the protein biosensors comprise at least one fusion protein comprising an antibody domain and a split reporter protein fragment domain (e.g., SmBiT, LgBiT). In some embodiments, the antibody domain is an scFv single chain antibody sequence, a Fab antibody fragment, and/or an immunoglobulin (e.g., IgG). In some embodiments, the antibody targets spike protein. In some embodiments, the antibody targets nucleocapsid protein.

Examples of fusion proteins comprising a split protein domain and an antibody domain include (FIG. 11): IgG-SmBiT; SmBiT-IgG (LC), where SmBiT is fused to the light chain of IgG; SmBiT-IgG (HC) where SmBiT is fused to the heavy chain of IgG; SmBiT-Fab (LC), where SmBiT is fused to the light chain of Fab; SmBiT-Fab (HC), where SmBiT is fused to the heavy chain of Fab; LgBiT-Fab (LC), where LgBiT is fused to the light chain of Fab; LgBiT-Fab (HC), where LgBiT is fused to the heavy chain of Fab; VH-SmBiT wherein VH is heavy chain variable region; IgG-LgBiT; LgBiT-IgG (LC), where LgBiT is fused to the light chain of IgG; LgBiT-IgG (HC) where LgBiT is fused to the heavy chain of IgG; VH-LgBiT wherein VH is heavy chain variable region, and scFv-LgBiT where scFv is a single chain Fv domain. The fusion proteins may form a dimer (e.g., a homodimer).

Exemplary fusion protein sequences for spike-binding antibody fusion proteins are provided in Table 4, full sequences are shown in the “Fusion protein” column, and domains in order from the amino-terminus to the carboxy-terminus are shown in the “Domains” column. In some embodiments, the Spike-binding antibodies include one of the HC sequences in Table 4 and a Spike-binding antibody LC sequence (e.g., SEQ ID NO: 12).

TABLE 4

Exemplary spike-binding antibody fusion protein sequences.

| | |
|---|---|
| Fusion protein | Domains |
| Spike HC-10 aa-SmBiT (SEQ ID NO:62) | Spike-binding antibody heavy chain (SEQ ID NO:13); human IgGl hinge and Fc domain (SEQ ID NO:47); linker (2 repeats of SEQ ID NO:34); SmBiT (SEQ ID NO:28) |
| Spike HC-10 aa-LgBiT (SEQ ID NO:63) | Spike-binding antibody heavy chain (SEQ ID NO:13); human IgGl hinge and Fc domain (SEQ ID NO:47); linker (2 repeats of SEQ ID NO:34); LgBiT (SEQ ID NO:29) |

Exemplary fusion protein sequences for nucleocapsid protein-binding antibody fusion proteins are provided in Table 5, where full sequences are shown in the “Fusion protein” column, and domains in order from the amino-terminus to the carboxy-terminus are shown in the “Domains” column. In some embodiments, the nucleocapsid protein-binding antibodies include one of the Fab HC sequences in the first two rows of Table 5 and a nucleocapsid protein-binding antibody LC sequence (e.g., SEQ ID NO:20). In some embodiments, the nucleocapsid protein-binding antibody fusion protein is an IgG antibody comprising a light chain sequence and a heavy chain sequence in the same row of Table 2. In some embodiments, the heavy chain sequence is part of a LgBiT or SmBiT fusion with the following domains in sequential order from the amino-terminus to the carboxy-terminus: heavy chain sequence; human IgGl hinge and Fc domain (SEQ ID NO:47); linker (2 repeats of SEQ ID NO:34); SmBiT (SEQ ID NO:28) OR LgBiT (SEQ ID NO:29).

TABLE 5

Exemplary nucleocapsid (N) protein-binding antibody fusion protein sequences.

| | |
|---|---|
| Fusion protein | Domains |
| N clone H2 Fab HC SmBiT (SEQ ID NO:81) | N clone H2 Fab heavy chain (SEQ ID NO:21); linker (SEQ ID NO:118); SmBiT (SEQ ID NO:28) |
| N clone H2 Fab HC LgBiT (SEQ ID NO:82) | N clone H2 Fab heavy chain (SEQ ID NO:21); linker (SEQ ID NO:119); LgBiT (SEQ ID NO:29); AviTag (amino acids 1-15 of SEQ ID NO:49) |
| N clone H2 scFv LC-HC SmBiT (SEQ ID NO:83) | N clone H2 light chain fragment (amino acids 1-109 of SEQ ID NO:20); linker (3 repeats of SEQ ID NO:34); N clone H2 heavy chain fragment (amino acids |
| | 1-126 of SEQ ID NO:21); linker (SEQ ID NO:34); SmBiT (SEQ ID NO:28); linker (SEQ ID NO:120); 8xHis tag (SEQ ID NO:51) |
| N clone H2 scFv LC-HC LgBiT (SEQ ID NO:84) | N clone H2 light chain fragment (amino acids 1-109 of SEQ ID NO:20); linker (3 repeats of SEQ ID NO:34); N clone H2 heavy chain fragment (amino acids 1-126 of SEQ ID NO:21); linker (SEQ ID NO:34); LgBiT (SEQ ID NO:29); linker (SEQ ID NO:120); 8xHis tag (SEQ ID NO:51) |
| N clone H2 scFv HC-LC SmBiT (SEQ ID NO:85) | N clone H2 heavy chain fragment (amino acids 1-126 of SEQ ID NO:21); linker (3 repeats of SEQ ID NO:34); N clone H2 light chain fragment (amino acids 1-109 of SEQ ID NO:20); linker (SEQ ID NO:34); SmBiT (SEQ ID NO:28); linker (SEQ ID NO:120); 8xHis tag (SEQ ID NO:51) |
| N clone H2 scFv HC-LC LgBiT (SEQ ID NO:86) | N clone H2 heavy chain fragment (amino acids 1-126 of SEQ ID NO:21); linker (3 repeats of SEQ ID NO:34); N clone H2 light chain fragment (amino acids 1-109 of SEQ ID NO:20); linker (SEQ ID NO:34); LgBiT (SEQ ID NO:29); linker (SEQ ID NO:120); 8xHis tag (SEQ ID NO:51) |

### VII. Detection Assays

In another aspect, provided herein are methods for detecting SARS-CoV virus in a test sample. As described in the Examples herein, the methods are able to detect SARS-CoV virus (e.g., SARS-CoV-2 virus) sensitively, quantitatively, and rapidly. In some embodiments, the test sample is a biological sample from a patient that may comprise SARS-CoV virus. The methods herein generally comprise using the protein biosensors described above in a mixture with the test sample, maintaining the mixture under conditions in which the protein biosensor detection moieties associate to produce an active reporter if the test sample comprises SARS-CoV virus, and detecting the active reporter, thereby determining that the test sample comprises SARS-CoV virus. In some embodiments, the SARS-CoV virus is SARS-CoV-2.

The methods provided herein are able to detect low amounts of SARS-CoV virus in a test sample. In some embodiments, the methods detect less than 1×10⁸ viral particles per mL, e.g., less than 1×10⁷ viral particles per mL, less than 1×10⁶ viral particles per mL, or less than 1×10⁵ particles per mL. In some embodiments, the methods detect SARS-CoV-2 at a concentration of less than 1×10⁸ viral particles per mL . In some embodiments, the lower limit of detection is greater than 100 viral particles per mL (e.g., 1×10³ viral particles per mL, 1×10⁴ viral particles per mL, or 1×10⁵ viral particles per mL).

### A. Producing a Mixture

The methods provided herein for detecting SARS-CoV virus in a test sample comprise producing a mixture by combining a) at least a portion of the test sample, b) a first fusion protein that comprises a first viral protein-binding domain and either a first peptide fragment of a split reporter protein or a first reporter moiety, and c) a second fusion protein that comprises a second viral protein-binding domain and either a second peptide fragment of the split reporter protein or a second reporter moiety. In some embodiments, the first viral protein-binding domain and the second viral protein-binding domain are each selected from the group consisting of an ACE2 polypeptide domain, a spike-binding antibody domain, and a nucleocapsid protein-binding antibody domain.

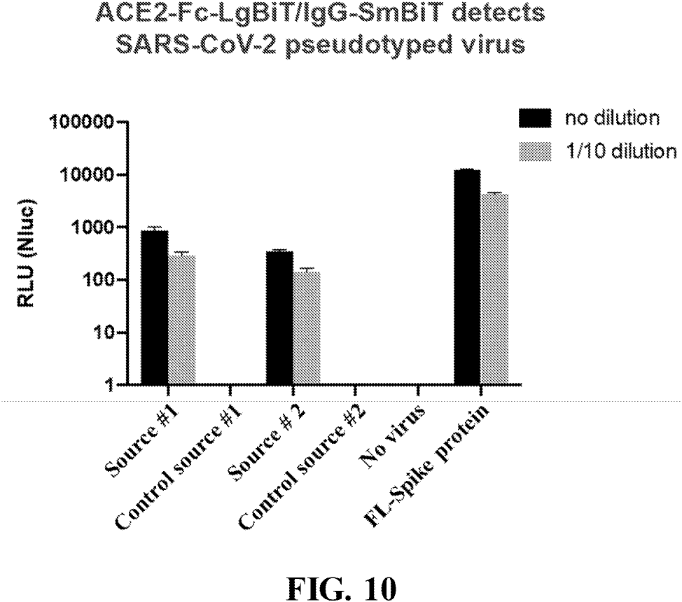

In some embodiments, each of the first viral protein-binding domain and the second viral protein-binding domain is an ACE2 polypeptide domain or a spike-binding antibody domain (see, e.g., Examples 5-8 and FIGS. 3-10). In these embodiments, each of the first viral protein-binding domain and the second viral protein-binding domain bind to the spike protein, as described above. In some embodiments, each of the first viral protein-binding domain and the second viral protein-binding domain are an ACE2 polypeptide domain (e.g., Examples 5 and 6 and FIGS. 3-8). In some embodiments, each of the first viral protein-binding domain and the second viral protein-binding domain are a spike-binding antibody domain. In some embodiments, the first viral protein-binding domain is an ACE2 polypeptide domain and the second viral protein-binding domain is a spike-binding antibody domain (e.g., Examples 7 and 8 and FIGS. 9 and 10). In some embodiments, the first viral protein-binding domain and the second viral protein-binding domain both bind to a first spike protein binding site. In some embodiments, the first viral protein-binding domain binds to the first spike protein binding site and the second viral protein-binding domain binds to a second spike protein binding site. In some embodiments, the first spike protein binding site and/or the second spike protein binding site are within a spike protein receptor binding domain (RBD). In some embodiments, the first spike protein binding site and/or the second spike protein binding site are not within a spike RBD. In some embodiments, the first viral protein-binding domain is an ACE2 polypeptide domain and the second viral protein-binding domain is a spike-binding antibody domain. In some embodiments, the spike-binding antibody domain specifically binds a binding site that is not within a spike RBD. In some embodiments, the spike-binding antibody domain specifically binds a binding site that is within a spike RBD. In some embodiments, the spike-binding antibody domain binding site within the RBD is not bound by the ACE2 polypeptide domain of the first viral protein-binding domain.

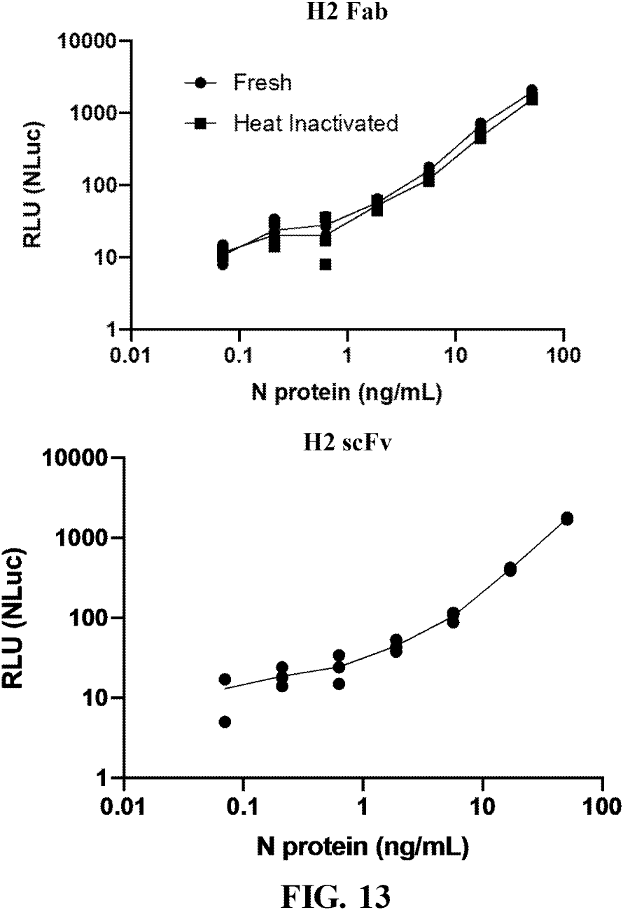

In some embodiments, both the first viral protein-binding domain and the second viral protein-binding domain is a nucleocapsid protein-binding antibody domain (see, e.g., Example 9 and FIGS. 12 and 13). In these embodiments, both the first viral protein-binding domain and the second viral protein-binding domain bind to the nucleocapsid protein, as described above. In some embodiments, the first viral protein-binding domain and the second viral protein-binding domain both bind to a first nucleocapsid protein binding site. In some embodiments, the first viral protein-binding domain binds to the first nucleocapsid protein binding site and the second viral protein-binding domain binds to a second nucleocapsid protein binding site. In some embodiments, the first viral protein-binding domain is an ACE2 polypeptide domain and the second viral protein-binding domain is a nucleocapsid protein-binding antibody domain. In some embodiments, the first viral protein-binding domain is a spike-binding antibody domain and the second viral protein-binding domain is a nucleocapsid protein-binding antibody domain.

In some embodiments, the first fusion protein comprises a dimerization domain. In some embodiments, the second fusion protein comprises a dimerization domain. In some embodiments, the dimerization domain comprises an antibody Fc domain. In some embodiments, the first and/or the second fusion protein comprises an antibody Fc domain and a viral protein-binding domain that is an ACE2 polypeptide domain. In some embodiments, the first and/or the second fusion protein comprises an antibody Fc domain and a viral protein-binding domain that is a spike-binding antibody domain. In some embodiments, the first and/or the second fusion protein comprises an antibody Fc domain and a viral protein-binding domain that is a nucleocapsid protein-binding antibody domain. In some embodiments, any of the fusion proteins that comprise an antibody Fc domain may be part of a fusion protein dimer. In some embodiments, a first fusion protein that comprises an antibody Fc domain is a subunit of a homodimer comprising monomers of the first fusion protein associated via an association between the antibody Fc domains. In some embodiments, the first fusion protein is a first subunit of a heterodimer and the second subunit of the heterodimer comprises a second antibody Fc domain that associates with the antibody Fc domain of the first fusion protein.

### B. Assay Conditions

The methods provided herein for detecting SARS-CoV virus in a test sample further comprise maintaining the mixture described above under conditions in which, only if the test sample comprises SARS-CoV virus, the detection moiety of the first and second fusion proteins come into sufficient proximity to associate (e.g., the first peptide fragment and the second peptide fragment associate to produce an enzymatically active reporter protein or the first reporter moiety and the second reporter moiety specifically associate). In some embodiments, if the test sample comprises SARS-CoV virus, the first fusion protein binds to a first viral protein on a virion and the second fusion protein binds to the first viral protein or to a second viral protein on the same virion. In some embodiments, the first viral protein and the second viral protein are any of the viral proteins expressed by the virus being detected (e.g., SARS-CoV-1, SARS-CoV-2). In some embodiments, the first and/or second viral protein is a spike protein (e.g., SARS-CoV-2 spike protein). In some embodiments, the first and/or second viral protein is a nucleocapsid protein (e.g., SARS-CoV-2 nucleocapsid protein).

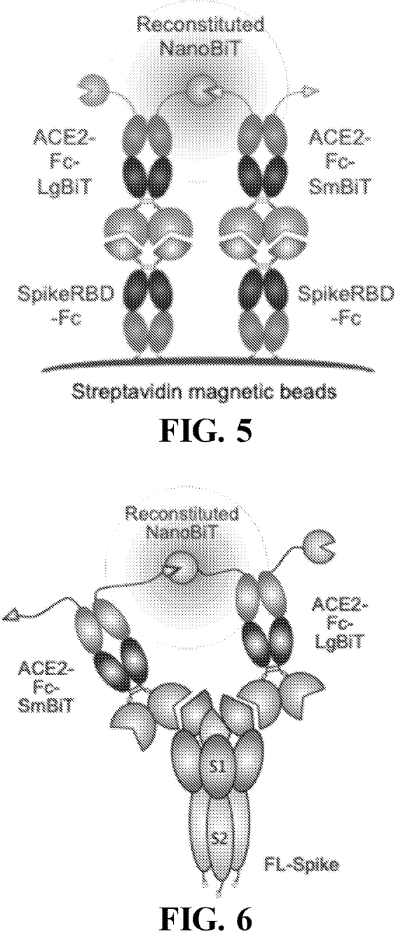

In some embodiments, the first peptide fragment and the second peptide fragment are fragments of an enzymatically active reporter protein (e.g., luciferase). In some embodiments, the association of the first peptide fragment and the second peptide fragment to produce the enzymatically active reporter protein comprises association of the first peptide fragment and the second peptide fragment (e.g., as shown in FIG. 3, top panel, FIG. 4, top panel, and FIGS. 5-7 and 9). In some embodiments, the association of the first peptide fragment and the second peptide fragment to produce the enzymatically active reporter protein comprises association of the first peptide fragment, the second peptide fragment, and a third peptide fragment of the reporter protein. In some embodiments, the third peptide fragment may be added to the reaction mixture with the detection reagents (e.g., as described below).

In some embodiments, the test sample is first diluted in a buffer before testing, i.e., to minimize interference from other components in the sample. In some embodiments, serial dilutions of the test sample are made to ensure at least some dilutions are within the dynamic range of the assay and to ensure accuracy. The dilution factor can be in the range of 1:1 to 1:50, e.g., between 1:2 and 1:40, between 1:5 and 1:35, or between 1:10 and 1:30, end points inclusive.

In some embodiments, the first fusion protein and the second fusion protein of the protein biosensor are present in the reaction mixture at approximately equal molar concentration to maximize the formation of the active reporter when virus is present. The term “approximately equal molar concentration,” refers to a difference between the molar concentrations of the two molecules of less than 30%, less than 20%, no greater than 10%, less than 5%, or less than 3% of the lesser value of the two molar concentrations. It is also desirable to maintain the protein biosensor concentration in the reaction mixture within an optimal range to obtain sufficiently high virus-specific signal while minimizing background readings. In some embodiments, a protein biosensor used in the assay, that is, each of the first and second fusion proteins of the protein biosensor, is present in a concentration that ranges from 0.1 nM to 10 nM, e.g., from 0.2 nM to 10 nM., from 0.3 nM to 3 nM, from 0.5 nM to 2 nM, or about 1 nM.

The test sample and the protein biosensors can be incubated under conditions suitable for the specific binding of the viral protein-binding domains to the virus (i.e., to viral proteins). The reaction and incubation are typically performed at ambient temperature, i.e., a temperature that is within the ranges of from 10° C. to 40° C., e.g., from 15° C. to 30° C., or from 18° C. to 25° C.

In some embodiments, the reaction mixture is maintained in a solution that has a pH less than 7.0 or less than 6.5 (e.g., pH 5-7, pH 5-6.5, or pH 5.5 to 6.5). A variety of buffers that have pH within this range and that are suitable for binding of the viral protein-binding domains to viral proteins can be used for methods disclosed herein, including buffers that are typically used for ELISA, e.g., PBS, TBS. In some approaches, the reaction mixture comprises Bovine Serum Albumin (BSA), Fetal Bovine Serum (FBS), and or TWEEN® 20, which are present in suitable amounts to minimize non-specific binding and reduce the background of the test.

In some embodiments, prior to the step of detecting the active reporter, the test sample may be incubated with the protein biosensors at ambient temperature for a period that is sufficient to allow the fusion protein viral protein-binding domains to bind to viral proteins, which will bring the two fusion protein detection moieties into proximity to form the active reporter, e.g., a luciferase protein or a hybridized nucleic acid reporter. In some embodiments, the length of incubation time ranges from 5 minutes to 1 hour (e.g., from 5 minutes to 10 minutes, from 5 minutes to 20 minutes, from 10 minutes to 30 minutes, from 10 minutes to 45 minutes, from 20 minutes to 40 minutes, from 20 minutes to 1 hour, or from 30 minutes to 1 hours). In some embodiments, the length of incubation time is 20 minutes.

The methods provided herein are generally easy to perform and amenable to use in a laboratory or a point-of-care setting equipped with basic liquid handling devices and appropriate detection systems (e.g., a luminescence plate reader or hand-held luminometer), as described below. In some embodiments, the assays herein can be performed in a small reaction volume (e.g., less than 50 µL) and in a high-throughput format (e.g., in a 384-well plate). In some embodiments, the assays herein only require 1 nM of each fusion protein.

### C. Detection

The methods provided herein for detecting SARS-CoV virus in a test sample further comprise detecting the association of the first peptide fragment and the second peptide fragment or the first reporter moiety and the second reporter moiety if the test sample comprises SARS-CoV virus. In some embodiments, the reaction mixture (i.e., as described above) comprises detection reagents. In some embodiments, the detection reagents are added to the reaction mixture along with the test sample and protein biosensor (i.e., the fusion proteins). In some embodiments, the detection reagents are added to the reaction mixture after incubation of the test sample and protein biosensor. In some embodiments, the reaction mixture may be incubated after addition of the detection reagents for a sufficient amount of time to allow the development of the detectable signal. The conditions for adding detection reagents, incubating the reaction mixture after adding detection reagents, and/or detecting the resulting signal may be selected based on the nature of the reporter (e.g., the split-protein or the nucleic acid reporter).

In some embodiments, the association of the first peptide fragment and the second peptide fragment (i.e., the first and second reporter moiety domains) or the first reporter moiety and the second reporter moiety can be detected based on enzymatic activity, probe amplification, or other split reporter methodologies. Such methodologies are well known and have been used for the detection and/or quantification of protein interactions. Many of the split reporter assays provided herein (e.g., the split-luciferase assay) are amenable for high-throughput runs with automation platforms. For example, a simulated run for 40 plates (3,840 assays) can be completed in 3 h on an automation workflow using the University of California, San Francisco (UCSF) Antibiome Center robotics platform. Serum sample transfer to an assay plate using Biomek Fx Automated Workstation may be completed in about 2 minutes. Robotics-assisted dispensing and luminescence reading for one iteration of 96 assays may take about 35 minutes.

In some embodiments, the reconstitution of the reporter protein (e.g., by the association of the first and second peptide fragments of the split reporter protein) produces an enzymatically active reporter that, in presence of suitable substrates and/or accessory reagents generates a detectable signal, as described above. Detectable signals include, without limitation, colorimetric, fluorescent and luminescent signals. In some embodiments, the substrate for the split reporter protein is luciferin, furimazine, or some other luminogenic substrate or molecule. In some embodiments, the reaction mixture comprises detection reagents and a detectable signal is produced by the enzymatically active reporter protein in the presence of the detection reagents.

In some embodiments, the split reporter is a luciferase protein (e.g., as described in Examples 5-9 and shown in FIGS. 3-10 and 13), and a substrate for the luciferase (e.g., coelenterazine, furimazine, luciferin, or some other luminogenic substrate) is added to the reaction mixture. After the addition of the substrate, the reaction mixture may be incubated for a sufficient amount of time to allow development of the signal. The step of signal development may last between 5 to 30 minutes (e.g., between 5 and 10 minutes, between 5 and 15 minutes, between 10 and 20 minutes, between 15 and 30 minutes, about 10 minutes, about 15 minutes, about 20 minutes, or about 25 minutes). In some embodiments, the step of signal development may last about 10 minutes.

In some embodiments, luminescent signals (e.g., signal produced by a reconstituted luciferase split-reporter protein) can be read by a luminescence microplate reader (e.g. Tecan Infinite 200 Pro, Promega GloMax), a portable luminometer (Junior LB9509), a hand-held ATP luminometer with customized sample tube (3M™ Clean-Trace™ Hygiene Monitoring and Management System), or a home-made luminometer to improve detection sensitivity and decrease required sample volume. In some embodiments, the luminescent signals can also be read using an app on a mobile phone or with an adaptor to a mobile phone camera.

In some embodiments, the first reporter moiety and the second reporter moiety are oligonucleotides that are partially complementary to each other. In some embodiments, the oligonucleotides are both partially complementary to an additional oligonucleotide (e.g., a splint oligonucleotide) in the reaction mixture. In some embodiments, the additional oligonucleotide is added to the reaction mixture as a detection reagent. In some embodiments, the reaction mixture comprises detection reagents and a detectable signal is produced by the specific association of the first and second reporter moieties (e.g., oligonucleotides) in the presence of the detection reagents. In some embodiments, the association between oligonucleotide reporter moieties (e.g., by hybridization) can be detected using proximity extension assays and/or proximity ligation assays, as described herein above.

### VIII. Kits

Materials and reagents useful for the diagnostic assays may be provided in kit form, optionally kits in which various reagents are provided in separate vials or containers. Kits may include fusion proteins comprising a binding moiety (e.g., an ACE2 domain as described herein) and a reporter moiety (e.g., a split reporter protein fragment as described herein); (ii) detection reagents (e.g., luciferin), and (iii) suitable buffers and other reagents. Multiple fusion proteins (e.g., an ACE2-SmBit fusion protein and an ACE2-LgBit fusion protein) may be provided in separate containers or premixed. In one approach, a kit may contain two recombinant protein reagents (binder1-SmBiT and binder2-LgBiT), substrates (such as luciferin), and an assay plate (such as white plate for luciferase assays).

### EXAMPLES

The following examples are offered to illustrate, but not to limit the claimed invention. Many of the following examples are further described in Lui, I., et al., 2020, “Trimeric SARS-CoV-2 Spike interacts with dimeric ACE2 with limited intra-Spike avidity,” bioRxiv, published May 21, 2020, doi:10.1101/2020.05.21.109157. Reference is made to this Lui et al., 2020 publication for illustration of certain experimental data as described in the instant disclosure.

### Example 1. Materials and Methods Used in Examples

Plasmids construction. Plasmids were constructed by standard molecular biology methods. The FL-Spike plasmid is described in (Amanat et al., 2020). The DNA fragments of Spike RBD, ACE2, LgBiT, were synthesized by IDT Technologies. The Spike-RBD-TEV-Fc-AviTag, ACE2-TEV-Fc-AviTag, Spike-RBD-8xHis-AviTag, ACE2-8xHis-AviTag plasmids were generated by subcloning the Spike-RBD or ACE2 DNA fragment into a pFUSE-hIgGl-Fc-AviTag vector (adapted from the pFUSE-hIgGl-Fc vector from InvivoGen). The ACE2-Fc-LgBiT plasmid was generated by subcloning the gene fragments of LgBiT to the N- or C-terminus of the ACE2-TEV-Fc-AviTag vector with a 10-amino acid (N-terminal fusion) or 5-amino acid (C-terminal fusion) linker to the ACE2 or Fc domains. The SmBiT tag in the ACE2-Fc-SmBiT plasmids were generated by overlap-extension PCR. The C-terminal AviTag was removed from all the ACE2-Fc reporter plasmids. Fab antibody fragment fusion proteins were expressed as described in Hornsby et al, 2015. scFv antibody fragment fusion proteins were expressed using a pFUSE vector (Invitrogen).

Expression and purification of ACE2 and Spike constructs. The ACE2 and Spike proteins were expressed and purified from Expi293 BirA cells according to established protocol from the manufacturer. Briefly, 30 µg of pFUSE (InvivoGen) vector encoding the protein of interest was transiently transfected into 75 million Expi293 BirA cells using the Expifectamine kit (Thermo Fisher Scientific). Enhancer was added 20 h after transfection. Cells were incubated for a total of 3 d at 37° C. in an 8% CO₂ environment before the supernatants were harvested by centrifugation. Fc-fusion proteins were purified by Protein A affinity chromatography and His-tagged proteins were purified by Ni-NTA affinity chromatography. Purity and integrity were assessed by SDS/PAGE. Purified protein was buffer exchanged into PBS and stored at -80° C. in aliquots.

Generation of ACE2 monomer. ACE2 monomer was obtained by TEV treatment of ACE2-Fc and subsequent purification. 50 µL Ni-NTA agarose (Qiagen) and 50 µL Neutravidin resin (Thermo Fisher Scientific) were washed with PBS-25 mM imidazole twice and combined in 100 µL PBS-25 mM imidazole. Next, 20 µg His-Tagged recombinant TEV protease and 1 mg purified ACE2-Fc protein were mixed, and the reaction tube was rotated at 4° C. for 30 minutes. The cleavage reaction was then incubated with the washed beads, rotating, at 4° C. for 30 minutes. While the incubation occurred, an additional 25 µL of magnetic Protein A beads and 25 µl or Ni-NTA beads were prepared as described before. Supernatant from the first bead clearance was transferred to the newly prepared beads and allowed to incubate for an additional 30 minutes at 4° C. To remove beads from the protein supernatant, reaction mixture was spin filtered at 1000 xg for 2 min and washed with an additional 250 uL of PBS-25 mM imidazole. The His-tagged TEV, biotinylated Fc, and the uncut ACE2-Fc remained on the beads while the monomeric ACE2 was isolated in the flowthrough. The purity of monomeric ACE2 was confirmed by SDS-PAGE electrophoresis. Purified protein was buffer exchanged to PBS and store at -80° C. in aliquots.

Differential scanning fluorimetry. To assess the stability of proteins, we measured the melting temperature (Tₘ) by doing differential scanning fluorimetry (DSF) as the method described previously (Hornsby et al., 2015). Briefly, purified protein was diluted to 0.5 µM or 0.25 µM in DSF buffer containing Sypro Orange 4x (Invitrogen) and PBS. 10 µL of reaction mixture was transferred to one well of a 384-well PCR plate. Duplicate was prepared as needed. In a Roche LC480 LightCycler, the reaction was heated from 30° C. to 95° C. with a ramp rate of 0.3° C. per 30 sec. The intensities of the fluorescent signal at an ~490 nm and ~575 nm (excitation and emission wavelengths) were continuously collected. The curve peak corresponds to the melting temperature of the protein. Data was processed and Tₘ was calculated using the Roche LC480 LightCycler software.

In vitro binding experiments. Biolayer interferometry data were measured using an Octet RED384 (ForteBio). Biotinylated Spike or Spike RBD protein were immobilized on the streptavidin (SA) biosensor. After blocking with biotin, purified ACE2 protein in solution was used as the analyte. PBS with 0.05% Tween-20 and 0.2% BSA was used for all diluents and buffers. A 1:1 monovalent binding model was used to fit the kinetic parameters (kₒₙ and koff).

Magnetic bead and solution based NanoBiT assays. For the Spike-Fc magnetic bead assay, magnetic beads were prepared by taking 100 µL of Streptavidin Magnesphere Paramagnetic Particles (Promega) and incubated with 5 µM of Spike-Fc-AviTag for 30 minutes, rotating at room temperature. Following, the beads were blocked with 10 µM biotin for 10 minutes. The beads were washed three times with PBS + 0.05% Tween + 0.2% BSA. 10 µL of 10-fold dilutions of the beads were incubated with 10 µL of premixed 2 nM ACE2-Fc-SmBiT and ACE2-Fc-LgBiT. The sample was incubated shaking at room temperature for 20 minutes. NanoGlo Luciferase substrate (Promega) diluted in NanoGlo Luciferase buffer was added to each well (15 µL) and luminescence was measured on a Tecan M1000 plate reader after 10 minutes.

For the detection of FL-Spike in solution, 10 µL of FL-Spike dilutions were combined with 10 µL of premixed 2 nM smBiT-ACE2-Fc and LgBiT-ACE2-Fc (FIG. 4), or 2 nM Spike-binding antibody IgG-SmBiT and ACE2-Fc-LgBiT (FIG. 9). Samples were incubated with substrate and luminescence was measured on a Tecan M1000 plate reader.

### Example 2. Structure Modeling of the Interaction Between Native ACE2 and SARS-CoV-2 Spike Trimer

Human ACE2 ectodomain is composed of a N-terminal peptidase domain (aa 18-614) and a C-terminal dimerization domain (aa 615-740). Cryo-EM structure of the full-length dimeric human ACE2 receptor in complex with Spike-RBD domain and an amino acid transporter B0AT1 has been determined (Yan et al., 2020). However, structures of SARS-CoV-2 FL-Spike in complex with either the full-length dimeric ACE2 or a monomeric ACE2 peptidase domain have not been reported, resulting in an incomplete understanding of the nature of this interaction.

To query if native ACE2 dimer can interact with Spike with intramolecular avidity, the structures of ACE2-Spike-RBD (PDB 6M17)(Yan et al., 2020) and FL-Spike (PDB 6VYB, “up-down-down” conformation)(Walls et al., 2020) were aligned (data not shown). Only one of the ACE2 peptidase domains is capable of accessing an RBD domain in the context of a Spike trimer, while the other ACE2 domain is oriented away from the other two RBD domains within the same Spike trimer.

In context of a therapeutic, it has been suggested that ACE2 (aa 18-614) monomer fused to dimeric human IgGl Fc (ACE2-Fc) is a potential therapeutic modality for blocking the host ACE2-viral Spike interaction (Lei et al., 2020; Li et al., 2020). In such a construct, the removal of the dimerization domain (aa 615-740) in ACE2, and the addition of linker and human IgG1 hinge allows for a more flexible positioning of the two ACE2 monomers. Modeling the interaction between FL-Spike and ACE2-Fc, one would expect to observe intramolecular avidity only if Spike-trimer can present two RBD domains in the “up” conformation for binding. While cryo-EM structures of SARS-CoV-2 Spike presenting zero (“three-down” closed conformation) or one (“one-up”) RBD domain has been reported, the “two-up” and “three-up” RBD conformations have only been reported for SARS-CoV-1 (Kirchdoerfer et al., 2018). Therefore, it remains unknown if these “two-up” or “three-up” conformations are present in SARS-CoV-2 Spike under physiological conditions for binding ACE2-Fc with intra-Spike avidity. Alternatively, if Spike-trimer presents on the viral surface at sufficient density, one would expect that ACE2-Fc could bind two separate Spike trimers, leading to inter-Spike avidity. However, the number and density of Spike proteins on the SARS-CoV-2 envelope remains unclear, in addition to whether there is heterogeneity between viral samples.

### Example 3. Design and Generation of Spike/ACE2 Constructs

A panel of Spike and ACE2 proteins in various multimeric formats were constructed to experimentally determine if ACE2-Fc binds with avidity to SARS-CoV-2 Spike (FIG. 1). The plasmid for expression of SARS-CoV-2 FL-Spike ectodomain (aa 1-1213) (FL-Spike) is described in (Amanat et al., 2020), and protein was expressed and purified as described (Amanat et al., 2020). In this plasmid, the sequence of the wildtype SARS-CoV-2 Spike ectodomain was modified to remove a furin cleavage site and to add a pair of stabilizing mutations. Additionally, a T4 trimerization motif and His6 tag were added to the C-terminus. Dimers of SARS-CoV-2 Spike Receptor Binding Domain (Spike-RBD) (328-533) and ACE2 (1-614) were designed as TEV-cleavable Fc-fusion molecules with a C-terminal AviTag™ (Avidity) for biotinylation (FIG. 1). Monomers of Spike-RBD (328-533) with a C-terminal TEV-His8-Avi were also generated. The human ACE2 ectodomain contains an N-terminal peptidase domain (aa 18-614) and a C-terminal dimerization domain (aa 615-740). The monomeric form of ACE2 (aa 18-614) (ACE2-monomer) was designed with a C-terminal TEV-8xHis-Avi tag, and the dimeric form of ACE2 (aa 18-614) (ACE2-Fc) was designed with a TEV-cleavable Fc-fusion molecule with a C-terminal Avi tag (Czajkowsky et al., 2012). All of the ACE2 and Spike proteins were expressed in BirA-ERexpressing Expi293 cells (Howarth et al., 2008; Martinko et al., 2018).

Proteins were purified by Protein A affinity chromatography for the Fc-fusion molecules and by Ni-NTA affinity chromatography for the monomeric molecules and FL-Spike protein. FL-Spike, Spike-RBD-Fc, Spike-RBD monomer, and ACE2-Fc all expressed at high yield and purity (see FIGS. 1B and 1C in Lui et al., 2020). However, ACE2 monomer did not express and was produced instead by TEV release from ACE2-Fc (FIG. 1, see FIG. 1B in Lui et al., 2020). All these proteins except for ACE2 monomer were >95% biotinylated during expression (see FIG. 1B in Lui et al., 2020), facilitating their use on avidin-functionalized surfaces and beads.

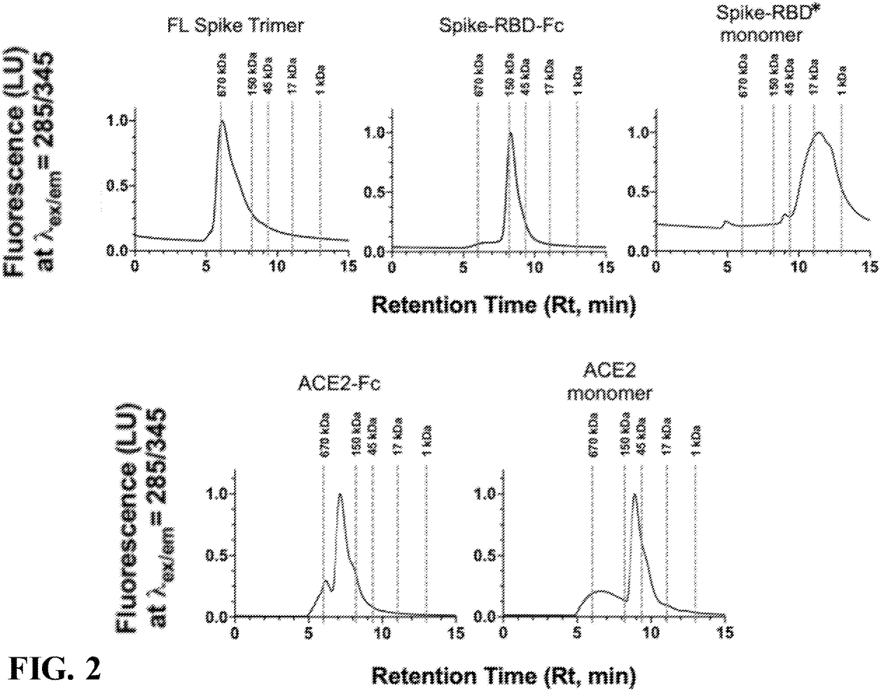

To confirm the oligomerization state of these proteins, size exclusion chromatography was performed. Trimeric FL-Spike, dimeric Spike-RBD-Fc, monomeric ACE2, dimeric ACE2-Fc all eluted at the expected elution time (FIG. 2), indicating successful generation of the different multimeric forms of Spike and ACE2 proteins. Spike-RBD monomer eluted later than expected, but further analysis of the associated SEC fractions by SDS-PAGE indicated pure protein at the correct molecular weight (see Supplementary Figure S1 in Lui et al., 2020). Differential scanning fluorimetry (DSF) of ACE2-Fc and ACE2 monomer showed these two proteins had similar Tₘ values (51.6 ± 0.1° C. for ACE2-Fc and 52.3 ± 0.5° C. for ACE2 monomer; see Supplementary Figure S2 in Lui et al., 2020).

### Example 4. Binding Characterization of ACE2 and Spike

The affinity and binding kinetics of the molecules described in Example 3 were determined by bio-layer interferometry (BLI) to understand how multimerization affects Spike and ACE2 interaction (Table 6; see FIG. 2 in Lui et al., 2020). Spike-RBD monomer, Spike-RBD-Fc and FL-Spike were immobilized on Streptavidin or Ni-NTA sensors, and ACE2 monomer or ACE2-Fc in solution were used as the analyte. A small (approximately four-fold) avidity effect was observed for the Spike-RBD/ACE2-Fc interaction (K_{D} = 5.5 nM, Table 6) compared to the Spike-RBD monomer/ACE2 monomer interaction (K_{D} = 18.5 nM, Table 6), which is likely due to local concentration effects. In contrast, the Spike-RBD-Fc/ACE2-Fc interaction (K_{D} < 10⁻¹² M, Table 6) showed a drastic increase (> 1000-fold) in binding affinity compared to the Spike-RBD-Fc/ACE2 monomer interaction (K_{D} =12.0 nM, Table 6). This dramatic increase in affinity is driven by a significant decrease in the off rate and little change in on rate, which indicates a strong intramolecular dimer-on-dimer (two-on-two) avidity between the two molecules.

TABLE 6

BLI-determined binding affinity and kinetics of Spike and ACE2 variants

| | | | | |
|---|---|---|---|---|
| Spike variant | ACE2 variant | K_{D} | kₒₙ (M⁻¹ sec⁻¹) | k_{off} (sec-¹) |
| Spike-RBD monomer | ACE2 monomer | 18.5 ± 0.038 nM | 2.7 x 10⁵ ± 5.1 x 10³ | 4.9 × 10⁻³ ± 3.1 × 10⁻ 5 |
| Spike-RBD Fc dimer | ACE2 monomer | 12.0 ± 0.028 nM | 3.6 × 10⁵ ± 7.9 × 10³ | 4.3 × 10⁻³ ± 3.3 × 10⁻ 5 |
| FL-Spike | ACE2 monomer | Not fitted | Not fitted | Not fitted |
| Spike-RBD monomer | ACE2-Fc dimer | 5.5 ± 0.048 nM | 2.9 × 10⁵ ± 2.0 × 10³ | 1.6 × 10⁻³ ± 8.7 × 10⁻ 6 |
| Spike-RBD Fc dimer | ACE2-Fc dimer | <1.0 × 10⁻¹² M | 1.2 x 10⁵ ± 1.2 x 10⁵ | <1.0 × 10⁻⁷ |
| FL-Spike | ACE2-Fc dimer | 5.9 ± 0.071 nM | 2.7 × 10⁴ ± 1.9 × 10² | 1.6 × 10⁻⁴ ± 1.6 × 10⁻ 6 |

Binding of ACE2 monomer and ACE2-Fc to FL-Spike were tested to determine the affinity and avidity effect. The binding interaction between ACE2 monomer and FL-Spike is very weak and could not be measured accurately (Table 6; see FIG. 2C in Lui et al., 2020). By contrast, the ACE2-Fc interacts with FL-Spike with a K_{D} of 5.9 nM, similar to the affinity for the isolated Spike-RBD-monomer (Table 6). This suggests that the presence of two ACE2 molecules in close proximity in ACE2-Fc is essential for a productive interaction with FL-Spike, and that a single ACE2 monomer is not sufficient to bind an RBD on FL-Spike. However, the affinity between FL-Spike/ACE2-Fc interaction was substantially less than Spike-RBD-Fc/ACE2-Fc interaction (K_{D} < 10⁻¹² M, Table 6), suggesting that the high-avidity two-on-two interaction is compromised in the context of FL-Spike. This could be due to geometric/steric constraints or the unique conformations of the RBDs in the FL-Spike context. When FL-Spike is loaded to a much higher density on the BLI sensor (loaded to 2.0 nm) and probed with ACE2-Fc, avidity can be recovered (see Supplementary Figure S3A in Lui et al., 2020). This indicates that if Spike is presented at high density, the ACE2-Fc arms can engage two RBDs (i.e., if neighboring Spike trimers are close enough). In contrast, monomeric ACE2 did not bind FL-Spike strongly even when FL-Spike was loaded until saturation, further demonstrating the importance of ACE2 dimerization for interacting with FL-Spike (see Supplementary Figure S3B in Lui et al., 2020).

The kₒₙ of the FL-Spike/ACE2-Fc interaction (Table 6; see FIG. 2 in Lui et al., 2020) is ~10-fold lower than the interactions between Spike-RBD-monomer or Spike-RBD-Fc with ACE2 or ACE2-Fc (Table 6), while the k_{off} is also ~10- to 20-fold lower. The decreased kₒₙ suggests that the RBDs in FL-Spike protein may have to undergo a conformational change for binding to ACE2. Previous cryo-EM studies on SARS-CoV-2 FL-Spike show that approximately half of the particles have the three RBD domains in the “down” conformation (Walls et al., 2020). Molecular dynamics simulations of SARS-CoV-2 FL-Spike suggest that the RBD exists in a series of conformations between the “down” and “up” states, and any RBD with an angle lower than 52.2° from the body of the trimer are inaccessible to ACE2 (Peng et al., 2020). The data herein support that a significant proportion of the RBDs in FL-Spike protein are in a “closed” or partially “closed” state inaccessible to ACE2, and the RBD has to open up to allow binding to ACE2. The decreased k_{off}, on the other hand, suggests that the presence of multiple RBDs within the context of a FLSpike could slow down the dissociation of ACE2-Fc.

A recent report on an antibody-bound structure of Spike-RBD demonstrates how ACE2 binding can lead to conformational changes that expose cryptic epitopes for antibody engagement (Yuan et al., 2020). However, it remains largely unknown exactly how ACE2 binding affects the conformation of FL-Spike because the structure of SARS-CoV-2 FL-Spike in complex with ACE2 has not been reported. A study of the EM structure of SARS-CoV-1 Spike in complex with ACE2 reported that the distribution of the RBD conformers was very different in the ACE2/Spike structure (28.1% “one-up”, 68% “two-up”, 3.9% “three-up”) compared to the Spike-only structure (58% “one-up”, 39% “two-up”, 3% “three-up”), which further supports our hypothesis that ACE2 binding may induce a conformational change in SARS-CoV-2 FL-Spike (Kirchdoerfer et al., 2018). However, caution should be exercised when comparing ACE2-binding datasets and conformational states between SARS-CoV-1 Spike and SARS-CoV-2 Spike, since the affinity of ACE2 to SARS-CoV-1 Spike is much weaker (K_{D} ~150 nM). Although the Spike proteins share relatively high sequence identity (~76%), small differences in sequence can account for dramatic changes in the protein conformational landscape.

### Example 5. ACE2-Fc-split Reporter Assay on FL-Spike Shows ACE2-Fc Binds FL-Spike With Limited Intra-Spike Avidity

To further investigate the RBD conformational landscape in FL-Spike, a split-luciferase system was designed to orthogonally probe the Spike/ACE2 interaction. ACE2-Fc reporter molecules where SmBiT or LgBiT were fused at the N- or C-termini were engineered, expressed, and purified (described in Example 1 above). All proteins were expressed in Expi293 cell with high yield and purity (see FIGS. 3B and 3C in Lui et al., 2020).

To functionally validate the split reporter system, Spike-RBD-Fc was immobilized on streptavidin magnetic beads at high-density. Incubation with 1 nM of ACE2-Fc-SmBiT and ACE2-Fc-LgBiT, or 1 nM of SmBiT-ACE2-Fc and LgBiT-ACE2-Fc with substrate (FIG. 3, top panel) showed dose-dependent luminescence signal, consistent with an assembled functional split enzyme reporter and intermolecular proximity (FIG. 3, bottom panel). The N-terminal fusion reporter pair showed higher sensitivity compared to the C-terminal fusion reporter pair (FIG. 5 and FIG. 6; see also Supplementary Figure S4 in Lui et al., 2020), suggesting that the increased entropy from the flexible linker and Fc domain reduces productive luciferase reconstitution.

The split reporters were used to interrogate the soluble FL-Spike trimer (FIG. 4). Increasing concentrations of soluble FL-Spike were incubated with ACE2-Fc-SmBiT/LgBiT or SmBiT/LgBiT-ACE2-Fc, followed by the addition of substrate (FIG. 4, top panel). The SmBiT/LgBiT-ACE2-Fc reporters showed dose-dependent increase in luminescence signal with 1-10 nM FL-Spike (FIG. 4, bottom panel). This result suggests that although FL-Spike cannot form a high-avidity two-on-two interaction with both arms of ACE2-Fc at a time, there is a proportion of FL-Spike proteins with “two-up” or perhaps even “three-up” RBDs that can simultaneously interact with multiple ACE2-Fc molecules. It is unclear if these “two-up” or “three-up” conformations are present prior to ACE2 binding, or appear because ACE2 binding induces a conformational change, which enables two or more of the RBD domains to engage in the binding to a second ACE2-Fc domain.

### Example 6. ACE2-Fc-split Reporter Assay to Detect Spike-RBD Domain on Magnetic Beads

Although the C-terminal ACE2-Fc-split reporter pair does not bind strongly to single FL-Spike protein, it can be used on live virus to interact with two adjacent Spike molecules and generate luciferase, because of the high density of Spike protein on SARS-CoV-2 viral surface (FIG. 7). As described above, Spike-RBD-Fc were immobilized on streptavidin magnetic beads, followed by incubation with C-terminal ACE2-Fc-SmBiT and ACE2-Fc-LgBiT (see schematic depiction in FIG. 5). Subsequent addition of the luciferase substrate demonstrated dose-dependent luminescence signal (FIG. 8). Similar experiments with N-terminal SmBiT-ACE2-Fc and LgBiT-ACE2-Fc also showed dose-dependent luminescence signal (see Example 5 and FIG. 3), suggesting that the ACE2-Fc split reporters may be able to detect live virus via interaction with two adjacent Spike molecules.

### Example 7. ACE2-Fc-split Reporter Assay Using anti-Spike Antibody to Detect Recombinant FL-Spike

To detect a single FL-Spike trimer a split reporter system was developed using ACE2-Fc-LgBiT and a Spike-binding IgG fusion protein (IgG-SmBiT). The IgG format of an anti-Spike antibody was used that interacts with Spike-RBD domain but does not compete with ACE2. IgG-SmBiT and ACE2-Fc-LgBiT fusions were engineered with a 5 amino acid linker between SmBiT/LgBiT with Fc (FIG. 9, top panel). IgG-SmBiT and ACE2-Fc-LgBiT were used as a split reporter against FL-Spike trimer in solution (FIG. 9, bottom panel). Increasing concentrations of FL-Spike (0.0001 - 10 nM) were incubated with 1 nM IgG-SmBiT and ACE2-Fc-LgBiT, followed by addition of luciferase substrate. IgG-SmBiT and ACE2-Fc-LgBiT generate dose-dependent luminescence signal against FL-Spike and detect as low as 3 pM FL-Spike in solution.

### Example 8. ACE2-Fc-split Reporter Assay Using anti-Spike Antibody to Detect SARS-CoV-2 Pseudotyped Lentivirus

The IgG-SmBiT and ACE2-Fc-LgBiT reporters can also detect SARS-CoV-2 pseudotyped virus (see, e.g., Crawford et al., 2020). Lentivirus expressing SARS-CoV-2 Spike protein or control VSV-G lentivirus at no dilution or 10-fold dilution were incubated with 1 nM IgG-SmBiT and ACE2-Fc-LgBiT, followed by addition of luciferase substrate. IgG-SmBiT and ACE2-Fc-LgBiT reporters generate luminescence signal only with SARS-CoV-2 pseudotyped virus (FIG. 10), demonstrating that the reporters can specifically detect SARS-CoV-2 Spike protein on live virus.

### Example 9 Anti-Nucleocapsid Protein Split Reporter Assay to Detect N Protein

As another approach for detecting SARS-CoV-2 virus, a split reporter system was developed to detect virus via binding of the split reporter fusion proteins to the SARS-CoV-2 nucleocapsid protein (N protein). Antibodies against the N protein were found by phage display selection against biotinylated N protein. Briefly, the biotinylated N protein was immobilized on streptavidin magnetic beads and four rounds of phage panning were performed with two Libraries (E and UCSF) simultaneously (for descriptions and use of libraries, see Miller et al., 2012 and Lim et al., 2021). They were then triaged via Fab-phage ELISA and the top clones were sequenced and cloned into the IgG scaffold (as described in Hornsby et al., 2015).

Six different antibody clones were tested in 36 different combinations of IgG SmBiT or LgBiT fusions for their ability to detect N protein. 1 nM of the SmBiT and LgBiT sensors were combined with decreasing concentrations of N protein (total volume 20 µL) and incubated at room temperature for 20 minutes. Then, 15 µL of NanoLuc substrate solution (Promega) was added to the sample, incubated for 10 minutes, and readout on a plate luminometer (data not shown). The top performing combination, clone H2, was further characterized for binding affinity and N protein detection.

The antibody sequences of clone H2 (IgG heavy chain sequence: SEQ ID NO:64; IgG light chain sequence: SEQ ID NO:20) were used to make different fusion protein formats (Fab and scFv) to test N protein detection (Fab heavy chain sequence: SEQ ID NO:21; Fab light chain sequence: SEQ ID NO:20). All of the Fab and scFv fragments denoted by H2 have the same CDR sequences (SEQ ID NOs: 15 and 23-27; see Table 2) that bind the N protein. For the Fab version, H2 Fab SmBiT and H2 Fab LgBiT were combined and used at 1 nM for detecting N protein. For the scFv version, two different linkages were made: one that has the LC variable domain N-terminally fused to the HC variable domain (LC-HC) and one that has the HC variable domain N-terminally fused to the LC variable domain (HC-LC). The LC-HC scFv SmBit and LC-HC scFv LgBiT were tested at 1 nM for their ability to detect the N protein.

BLI was used to determine the binding affinity of H2 Fab for N protein. Briefly, the N protein was immobilized on a streptavidin sensor and incubated with various concentrations of the H2 Fab to determine the binding affinity of the Fab (FIG. 12). The binding affinity was determined to be 20 nM.

The H2 Fab SmBiT/LgBiT sensor combination and H2 LC-HC scFv SmBiT/LgBiT sensor combinations were tested for ability to detect purified N protein in solution. The sensors were used at 1 nM and incubated with various concentrations of the N protein for 20 minutes at room temperature, followed by addition of NanoLuc substrate, 10 minute incubation, and readout on a luminometer for 1 second. Both sensor combinations were able to detect SARS-CoV-2 N protein with a limit of detection (LOD) of approximately 1.9 ng/mL (FIG. 13) The Fab sensors were also able to detect heat inactivated N protein (FIG. 13). The LOD for the N protein biosensors is much lower than the widely used BinaxNOW Abbott antigen detection assay, which is approximately 20 ng/mL (see, e.g., Pilarowski et al., 2021).

References cited in the Examples:

Amanat, et al., 2020, “A serological assay to detect SARS-CoV-2 seroconversion in humans,” Nat. Med. 26:1033-1036.

Crawford, et al., 2020, “Protocol and reagents for pseudotyping lentiviral particles with SARS-CoV-2 spike protein for neutralization assays,” Viruses 12:513.

Czajkowsky, et al., 2012, “Fc-fusion proteins: New developments and future perspectives,” EMBO Molecular Medicine, 4(10), 1015-1028, doi:10.1002/emmm.201201379.

Hornsby, et al., 2015, “A High Through-put Platform for Recombinant Antibodies to Folded Proteins. Molecular & Cellular Proteomics,” 14(10):2833-2847.

Howarth, et al., 2008, “Monovalent, reduced-size quantum dots for imaging receptors on living cells,” Nature Methods 5(5):397-399.

Kirchdoerfer, et al., 2018, “Stabilized coronavirus spikes are resistant to conformational changes induced by receptor recognition or proteolysis,” Scientific Reports 8:15701, doi:10.1038/s41598-018-34171-7.

Lei, et al., 2020, “Potent neutralization of 2019 novel coronavirus by recombinant ACE2-Ig,” bioRxiv, published Feb. 3, 2020, doi:10.1101/2020.02.01.929976.

Li, et al., 2020, “SARS-CoV-2 and Three Related Coronaviruses Utilize Multiple ACE2 Orthologs and Are Potently Blocked by an Improved ACE2-Ig,” Journal of Virology 94(22):e01283-20, doi:10.1128/JVI.01283-20.

Lim et al., 2021, “Bispecific VH/Fab antibodies targeting neutralizing and non-neutralizing Spike epitopes demonstrate enhanced potency against SARS-CoV-2,” mAbs 13(1), doi:10.1080/19420862.2021.1893426

Lui, I., et al., 2020, “Trimeric SARS-CoV-2 Spike interacts with dimeric ACE2 with limited intra-Spike avidity,” bioRxiv, published May 21, 2020, doi:10.1101/2020.05.21.109157.

Martinko, et al., 2018, “Targeting RAS-driven human cancer cells with antibodies to upregulated and essential cell-surface proteins,” ELife 7: e31098, doi:10.7554/eLife.31098.

Miller et al., 2012, “T cell receptor-like recognition of tumor in vivo by synthetic antibody fragment,” PLoS One 7(8):e43746

Peng, et al., 2020, “Exploring the Binding Mechanism and Accessible Angle of SARS-CoV-2 Spike and ACE2 by Molecular Dynamics Simulation and Free Energy Calculation,” chemrxiv.org, published Feb. 21, 2020, doi:10.26434/chemrxiv.11877492.v1.

Pilarowski et al., 2021, “Performance characteristics of a rapid severe acute respiratory syndrome coronavirus 2 antigen detection assay at a public plaza testing site in San Francisco,” Journal of Infect. Dis. 223(7):1129-1144

Walls, et al., 2020, “Structure, Function, and Antigenicity of the SARS-CoV-2 Spike Glycoprotein,” Cell 180:281-292, doi:10.1016/j.cell.2020.02.058.

Yan, et al., 2020, “Structural basis for the recognition of SARS-CoV-2 by full-length human ACE2,” Science 367(6485):1444-1448, doi:10.1126/science.abb2762.

Yuan, et al., 2020, “A highly conserved cryptic epitope in the receptor-binding domains of SARS-CoV-2 and SARS-CoV,” Science 368(6491):630-633, doi:10.1126/science.abb7269.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

The following copending commonly owned patent applications are incorporated by reference in their entirety for all purposes:
DETECTION ASSAY FOR ANTI-SARS-COV-2 ANTIBODIES, Application No. PCT/US , filed May 11, 2021 (attorney docket number 103182-1244658-005510WO), and
ACE2 COMPOSITIONS AND METHODS, Application No. PCT/US, filed May 11, 2021 (attorney docket number 103182-1244650-005010WO).

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

1. A method for detecting SARS-CoV virus in a test sample comprising:
i) producing a mixture by combining
a) at least a portion of the test sample;
b) a first fusion protein that comprises a first viral protein-binding domain and either a first peptide fragment of a split reporter protein or a first reporter moiety; and
c) a second fusion protein that comprises a second viral protein-binding domain and either a second peptide fragment of the split reporter protein or a second reporter moiety;
ii) maintaining the mixture under conditions in which, only if the test sample comprises SARS-CoV virus, the first peptide fragment and the second peptide fragment associate to produce an enzymatically active reporter protein or the first reporter moiety and the second reporter moiety specifically associate; and
iii) detecting the association of the first peptide fragment and the second peptide fragment or the first reporter moiety and the second reporter moiety if the test sample comprises SARS-CoV virus.

2. The method of claim 1, wherein the first viral protein-binding domain and the second viral protein-binding domain are each selected from the group consisting of an ACE2 polypeptide domain, a spike-binding antibody domain, and a nucleocapsid protein-binding antibody domain.

3. The method of claim 1, wherein each of the first viral protein-binding domain and the second viral protein-binding domain is an ACE2 polypeptide domain or a spike-binding antibody domain, and wherein:
(i) the first viral protein-binding domain and the second viral protein-binding domain both bind to a first spike protein binding site, or
(ii) the first viral protein-binding domain binds to the first spike protein binding site and the second viral protein-binding domain binds to a second spike protein binding site.

4. The method of claim 3, wherein the first spike protein binding site and/or the second spike protein binding site are within a spike protein receptor binding domain (RBD).

5. The method of claim 3, wherein the first spike protein binding site and/or the second spike protein binding site are not within a spike protein RBD.

6. The method of claim 2, wherein each of the first viral protein-binding domain and the second viral protein-binding domain is a nucleocapsid protein-binding antibody domain, and wherein:
(i) the first viral protein-binding domain and the second viral protein-binding domain both bind to a first nucleocapsid protein binding site, or
(ii) the first viral protein-binding domain binds to the first nucleocapsid protein binding site and the second viral protein-binding domain binds to a second nucleocapsid protein binding site.

7. The method of claim 2, wherein the first fusion protein and/or the second fusion protein further comprise a dimerization domain.

8. The method of claim 7, wherein the dimerization domain comprises an antibody Fc domain.

9. The method of claim 1 wherein, if the test sample comprises SARS-CoV virus, the first fusion protein binds to a first viral protein on a virion and the second fusion protein binds to the first viral protein or to a second viral protein on the same virion.

10. The method of claim 9, wherein the first viral protein and the second viral protein are each selected from the group consisting of a spike protein and a nucleocapsid protein.

11. The method of claim 1, wherein the mixture comprises detection reagents and a detectable signal is produced by the action of the enzymatically active reporter protein in the presence of the detection reagents.

12. The method of claim 1, wherein in the association in step (ii) to produce the enzymatically active reporter protein comprises association of the first peptide fragment, the second peptide fragment and a third peptide fragment of the reporter protein.

13. The method of claim 1, in which the reporter protein is luciferase.

14. The method of claim 1, wherein the first reporter moiety and the second reporter moiety are oligonucleotides that
i) are partially complementary to each other, or
ii) are both partially complementary to an oligonucleotide in the mixture.

15. The method of claim 14, wherein the mixture comprises detection reagents and a detectable signal is produced by the specific association of the first and second reporter moieties in the presence of the detection reagents.

16. The method of claim 1, wherein the SARS-CoV virus is SARS-CoV-2.

17. The method of claim 16 wherein SARS-CoV-2 is detected at a concentration of less than 1x108 viral particles per mL.

18. A fusion protein that comprises an RBD-binding ACE2 polypeptide domain and a first peptide fragment of a split reporter protein or a first reporter moiety.

19. A fusion protein that comprises a spike-binding antibody domain and a peptide fragment of a split reporter protein or a reporter moiety.

20. A fusion protein that comprises a nucleocapsid protein-binding antibody domain and a peptide fragment of a split reporter protein or a reporter moiety.

21. The fusion protein of claim 18, further comprising a dimerization domain.

22. A composition comprising two fusion proteins, wherein each fusion protein is the fusion protein of claim 18 and i) the split reporter proteins are complementary fragments of a reporter protein or ii) the reporter moieties are oligonucleotides that are partially complementary to each other or are both partially complementary to an additional oligonucleotide.


SEQUENCE LISTING (120 sequences)

SEQ ID NO: 1 - protein, 805 aa, Homo sapiens [sequence omitted]

SEQ ID NO: 2 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 597 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 4 - protein, 126 aa, Homo sapiens
DQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 5 - protein, 614 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 6 - protein, 740 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 7 - protein, 740 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 8 - protein, 729 aa, Artificial Sequence
  Synthetic construct
MSSSSWLLLSLVAVTAAQSTIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQP

SEQ ID NO: 9 - protein, 206 aa, Severe acute respiratory syndrome coronavirus 2
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 10 - protein, 162 aa, Severe acute respiratory syndrome coronavirus 2
PRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 137 aa, Severe acute respiratory syndrome coronavirus 2
GLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGS

SEQ ID NO: 12 - protein, 215 aa, Artificial Sequence
  Synthetic construct
DIQMTQSPSSLSASVGDRVTITCRASQSVGSALAWYQQKPGKAPKLLIYSASSLYSGVPSRFSGSRSGTDFTLTISSLQPEDFATYYCQQYAYSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 216 aa, Artificial Sequence
  Synthetic construct
EVQLVESGGGLVQPGGSLRLSCAASGFNIYSSYMHWVRQAPGKGLEWVAYISPYYGSTYYADSVKGRFTISRDNSKNTLYLQMNSLRAEDTAVYYCARYVAGYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 14 - protein, 7 aa, Artificial Sequence
  Synthetic construct
SVGSALA

SEQ ID NO: 15 - protein, 6 aa, Artificial Sequence
  Synthetic construct
SASSLY

SEQ ID NO: 16 - protein, 9 aa, Artificial Sequence
  Synthetic construct
QYAYSSLIT

SEQ ID NO: 17 - protein, 8 aa, Artificial Sequence
  Synthetic construct
NIYSSYMH

SEQ ID NO: 18 - protein, 12 aa, Artificial Sequence
  Synthetic construct
AYISPYYGSTYY

SEQ ID NO: 19 - protein, 10 aa, Artificial Sequence
  Synthetic construct
ARYVAGYGFD

SEQ ID NO: 20 - protein, 215 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQFIQFPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 234 aa, Artificial Sequence
  Synthetic construct
EISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 22 - protein, 256 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQFIQFPITFGQGTKVEIKRGGGGSGGGGSGGGGSEISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSHHHHHH

SEQ ID NO: 23 - protein, 7 aa, Artificial Sequence
  Synthetic construct
SVSSAVA

SEQ ID NO: 24 - protein, 8 aa, Artificial Sequence
  Synthetic construct
QFIQFPIT

SEQ ID NO: 25 - protein, 8 aa, Artificial Sequence
  Synthetic construct
NVYSSSIH

SEQ ID NO: 26 - protein, 12 aa, Artificial Sequence
  Synthetic construct
ASISSYYSYTSY

SEQ ID NO: 27 - protein, 15 aa, Artificial Sequence
  Synthetic construct
ARSYNFSWSGYYGFD

SEQ ID NO: 28 - protein, 11 aa, Artificial Sequence
  Synthetic construct
VTGYRLFEEIL

SEQ ID NO: 29 - protein, 158 aa, Artificial Sequence
  Synthetic construct
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 30 - protein, 22 aa, Artificial Sequence
  Synthetic construct
GSSGGGGSGGGGSGGGGSGGGG

SEQ ID NO: 31 - protein, 17 aa, Artificial Sequence
  Synthetic construct
GSSGGGGSGGGGSGGGG

SEQ ID NO: 32 - protein, 12 aa, Artificial Sequence
  Synthetic construct
GSSGGGGSGGGG

SEQ ID NO: 33 - protein, 11 aa, Artificial Sequence
  Synthetic construct
CSGGGGSGGGG

SEQ ID NO: 34 - protein, 5 aa, Artificial Sequence
  Synthetic construct
GGGGS

SEQ ID NO: 35 - protein, 6 aa, Artificial Sequence
  Synthetic construct
GSSGSS

SEQ ID NO: 36 - protein, 15 aa, Artificial Sequence
  Synthetic construct
SGSETPGTSESATPE

SEQ ID NO: 37 - protein, 16 aa, Artificial Sequence
  Synthetic construct
SGSETPGTSESATPES

SEQ ID NO: 38 - protein, 15 aa, Artificial Sequence
  Synthetic construct
GGGGSGGGGSGGGGS

SEQ ID NO: 39 - protein, 25 aa, Artificial Sequence
  Synthetic construct
GGGGSGGGGSGGGGSGGGGSGGGGS

SEQ ID NO: 40 - protein, 50 aa, Artificial Sequence
  Synthetic construct
GGGGSGGGGSGGGGSGGGGSGGGGSGGGGSGGGGSGGGGSGGGGSGGGGS

SEQ ID NO: 41 - protein, 8 aa, Artificial Sequence
  Synthetic construct
GGGGGGGG

SEQ ID NO: 42 - protein, 12 aa, Artificial Sequence
  Synthetic construct
GSAGSAAGSGEF

SEQ ID NO: 43 - protein, 17 aa, Artificial Sequence
  Synthetic construct
AEAAAKEAAAKEAAAKA

SEQ ID NO: 44 - protein, 52 aa, Artificial Sequence
  Synthetic construct
AEAAAKEAAAKEAAAKEAAAKEAAAKEAAAKEAAAKEAAAKEAAAKEAAAKA

SEQ ID NO: 45 - protein, 201 aa, Homo sapiens
PKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 31 aa, Homo sapiens
EPKSCDKTHTCPPCPAPELLGGPSVFLFPPK

SEQ ID NO: 47 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 227 aa, Artificial Sequence
  Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 16 aa, Artificial Sequence
  Synthetic construct
GLNDIFEAQKIEWHEG

SEQ ID NO: 50 - protein, 10 aa, Artificial Sequence
  Synthetic construct
EQKLISEEDL

SEQ ID NO: 51 - protein, 8 aa, Artificial Sequence
  Synthetic construct
HHHHHHHH

SEQ ID NO: 52 - protein, 19 aa, Artificial Sequence
  Synthetic construct
MRMQLLLLIALSLALVTNS

SEQ ID NO: 53 - protein, 7 aa, Artificial Sequence
  Synthetic construct
ENLYFQS

SEQ ID NO: 54 - protein, 22 aa, Artificial Sequence
  Synthetic construct
TSSGGGGENLYFQSSGGGSGGG

SEQ ID NO: 55 - protein, 886 aa, Artificial Sequence
  Synthetic construct
TSMSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSVTGYRLFEEIL

SEQ ID NO: 56 - protein, 1033 aa, Artificial Sequence
  Synthetic construct
TSMSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 57 - protein, 1043 aa, Artificial Sequence
  Synthetic construct
TSMSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 58 - protein, 1053 aa, Artificial Sequence
  Synthetic construct
TSMSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 59 - protein, 1063 aa, Artificial Sequence
  Synthetic construct
TSMSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 60 - protein, 899 aa, Artificial Sequence
  Synthetic construct
TSLEVTGYRLFEEILGGGGSGGGGSLEQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEG

SEQ ID NO: 61 - protein, 1046 aa, Artificial Sequence
  Synthetic construct
TSLEVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGGSGGGGSLEQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYATSSGGGGENLYFQSSGGGSGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGLNDIFEAQKIEWHEG

SEQ ID NO: 62 - protein, 471 aa, Artificial Sequence
  Synthetic construct
TSEVQLVESGGGLVQPGGSLRLSCAASGFNIYSSYMHWVRQAPGKGLEWVAYISPYYGSTYYADSVKGRFTISRDNSKNTLYLQMNSLRAEDTAVYYCARYVAGYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSVTGYRLFEEIL

SEQ ID NO: 63 - protein, 618 aa, Artificial Sequence
  Synthetic construct
TSEVQLVESGGGLVQPGGSLRLSCAASGFNIYSSYMHWVRQAPGKGLEWVAYISPYYGSTYYADSVKGRFTISRDNSKNTLYLQMNSLRAEDTAVYYCARYVAGYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 64 - protein, 221 aa, Artificial Sequence
  Synthetic construct
EVQLVESGGGLVQPGGSLRLSCAASGFNVYSSSIHWVRQAPGKGLEWVASISSYYSYTSYADSVKGRFTISRDNSKNTLYLQMNSLRAEDTAVYYCARSYNFSWSGYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 65 - protein, 215 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSWSEPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 217 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYHKHKKPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 216 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYWHQYPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 216 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYQSSPPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 215 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYYFPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 216 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYKKLGPITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 215 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQYGSYLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 215 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSYWWPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 223 aa, Artificial Sequence
  Synthetic construct
EVQLVESGGGLVQPGGSLRLSCAASGFNVSYYYIHWVRQAPGKGLEWVASIYSYSGYTSYADSVKGRFTISRDNSKNTLYLQMNSLRAEDTAVYYCARDEIYGYWTGWQEGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 74 - protein, 225 aa, Artificial Sequence
  Synthetic construct
EVQLVESGGGLVQPGGSLRLSCAASGFNFYSYSIHWVRQAPGKGLEWVAYISSSYGYTSYADSVKGRFTISRDNSKNTLYLQMNSLRAEDTAVYYCARSPYGYSRKSYLYWSAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 75 - protein, 227 aa, Artificial Sequence
  Synthetic construct
EVQLVESGGGLVQPGGSLRLSCAASGFNIYSSSIHWVRQAPGKGLEWVASIYSYYGSTSYADSVKGRFTISRDNSKNTLYLQMNSLRAEDTAVYYCARDFYLHMYGYPIYSQYEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 76 - protein, 227 aa, Artificial Sequence
  Synthetic construct
EVQLVESGGGLVQPGGSLRLSCAASGFNFSSYYIHWVRQAPGKGLEWVASIYPSSSSTSYADSVKGRFTISRDNSKNTLYLQMNSLRAEDTAVYYCARSTSSGRYYGGSFSYMYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 77 - protein, 227 aa, Artificial Sequence
  Synthetic construct
EVQLVESGGGLVQPGGSLRLSCAASGFNIYSSSIHWWVRQAPGKGLEWVASISSYYGSTSYADSVKGRFTISRDNSKNTLYLQMNSLRAEDTAVYYCARHPHYVGQYWGWYESYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 78 - protein, 227 aa, Artificial Sequence
  Synthetic construct
EVQLVESGGGLVQPGGSLRLSCAASGFNVYSSSIHWVRQAPGKGLEWVASIYSSYGYTSYADSVKGRFTISRDNSKNTLYLQMNSLRAEDTAVYYCARSSRVWFYSWKPYRYNRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 79 - protein, 225 aa, Artificial Sequence
  Synthetic construct
EVQLVESGGGLVQPGGSLRLSCAASGFNVSSSYIHWVRQAPGKGLEWVASIYSYYGYTSYADSVKGRFTISRDNSKNTLYLQMNSLRAEDTAVYYCARYHFQYWKHWIWFSGALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 80 - protein, 224 aa, Artificial Sequence
  Synthetic construct
EVQLVESGGGLVQPGGSLRLSCAASGFNFSSYSIHWVRQAPGKGLEWVAYISSSSGYTYYADSVKGRFTISRDNSKNTLYLQMNSLRAEDTAVYYCARETYSQYHYGWFYQGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 81 - protein, 253 aa, Artificial Sequence
  Synthetic construct
EISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGGVTGYRLFEEIL

SEQ ID NO: 82 - protein, 419 aa, Artificial Sequence
  Synthetic construct
EISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSAGGLNDIFEAQKIEWHE

SEQ ID NO: 83 - protein, 279 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQFIQFPITFGQGTKVEIKRGGGGSGGGGSGGGGSEISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSGGGGSVTGYRLFEEILGGGSGHHHHHHHH

SEQ ID NO: 84 - protein, 426 aa, Artificial Sequence
  Synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQFIQFPITFGQGTKVEIKRGGGGSGGGGSGGGGSEISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHH

SEQ ID NO: 85 - protein, 279 aa, Artificial Sequence
  Synthetic construct
EISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSGGGGSGGGGSGGGGSSDIQMTQSPSSLSASVGDRVTITCRASQSVSSAVAWYQQKPGKAPKLLIYSASSLYSGVPSRFSGSRSGTDFTLTISSLQPEDFATYYCQQFIQFPITFGQGTKVEIKRGGGGSVTGYRLFEEILGGGSGHHHHHHHH

SEQ ID NO: 86 - protein, 426 aa, Artificial Sequence
  Synthetic construct
EISEVQLVESGGGLVQPGGSLRLSCAASGFNVYSSSIHWVRQAPGKGLEWVASISSYYSYTSYADSVKGRFTISADTSKNTAYLQMNSLRAEDTAVYYCARSYNFSWSGYYGFDYWGQGTLVTVSSGGGGSGGGGSGGGGSSDIQMTQSPSSLSASVGDRVTITCRASQSVSSAVAWYQQKPGKAPKLLIYSASSLYSGVPSRFSGSRSGTDFTLTISSLQPEDFATYYCQQFIQFPITFGQGTKVEIKRGGGGSVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSGHHHHHHHH

SEQ ID NO: 87 - protein, 8 aa, Artificial Sequence
  Synthetic construct
QSWSEPLT

SEQ ID NO: 88 - protein, 8 aa, Artificial Sequence
  Synthetic construct
NVSYYYIH

SEQ ID NO: 89 - protein, 12 aa, Artificial Sequence
  Synthetic construct
ASIYSYSGYTSY

SEQ ID NO: 90 - protein, 17 aa, Artificial Sequence
  Synthetic construct
ARDEIYGYWTGWQEGMD

SEQ ID NO: 91 - protein, 10 aa, Artificial Sequence
  Synthetic construct
QYHKHKKPIT

SEQ ID NO: 92 - protein, 8 aa, Artificial Sequence
  Synthetic construct
NFYSYSIH

SEQ ID NO: 93 - protein, 12 aa, Artificial Sequence
  Synthetic construct
AYISSSYGYTSY

SEQ ID NO: 94 - protein, 19 aa, Artificial Sequence
  Synthetic construct
ARSPYGYSRKSYLYWSAMD

SEQ ID NO: 95 - protein, 9 aa, Artificial Sequence
  Synthetic construct
QYWHQYPVT

SEQ ID NO: 96 - protein, 8 aa, Artificial Sequence
  Synthetic construct
NIYSSSIH

SEQ ID NO: 97 - protein, 12 aa, Artificial Sequence
  Synthetic construct
ASIYSYYGSTSY

SEQ ID NO: 98 - protein, 21 aa, Artificial Sequence
  Synthetic construct
ARDFYLHMYGYPIYSQYEGFD

SEQ ID NO: 99 - protein, 9 aa, Artificial Sequence
  Synthetic construct
QYQSSPPIT

SEQ ID NO: 100 - protein, 8 aa, Artificial Sequence
  Synthetic construct
NFSSYYIH

SEQ ID NO: 101 - protein, 12 aa, Artificial Sequence
  Synthetic construct
ASIYPSSSSTSY

SEQ ID NO: 102 - protein, 21 aa, Artificial Sequence
  Synthetic construct
ARSTSSGRYYGGSFSYMYGLD

SEQ ID NO: 103 - protein, 8 aa, Artificial Sequence
  Synthetic construct
QSYYFPIT

SEQ ID NO: 104 - protein, 9 aa, Artificial Sequence
  Synthetic construct
NIYSSSIHW

SEQ ID NO: 105 - protein, 12 aa, Artificial Sequence
  Synthetic construct
ASISSYYGSTSY

SEQ ID NO: 106 - protein, 20 aa, Artificial Sequence
  Synthetic construct
ARHPHYVGQYWGWYESYGLD

SEQ ID NO: 107 - protein, 9 aa, Artificial Sequence
  Synthetic construct
QYKKLGPIT

SEQ ID NO: 108 - protein, 12 aa, Artificial Sequence
  Synthetic construct
ASIYSSYGYTSY

SEQ ID NO: 109 - protein, 21 aa, Artificial Sequence
  Synthetic construct
ARSSRVWFYSWKPYRYNRGMD

SEQ ID NO: 110 - protein, 8 aa, Artificial Sequence
  Synthetic construct
QYGSYLIT

SEQ ID NO: 111 - protein, 8 aa, Artificial Sequence
  Synthetic construct
NVSSSYIH

SEQ ID NO: 112 - protein, 12 aa, Artificial Sequence
  Synthetic construct
ASIYSYYGYTSY

SEQ ID NO: 113 - protein, 19 aa, Artificial Sequence
  Synthetic construct
ARYHFQYWKHWIWFSGALD

SEQ ID NO: 114 - protein, 8 aa, Artificial Sequence
  Synthetic construct
QSYWWPVT

SEQ ID NO: 115 - protein, 8 aa, Artificial Sequence
  Synthetic construct
NFSSYSIH

SEQ ID NO: 116 - protein, 12 aa, Artificial Sequence
  Synthetic construct
AYISSSSGYTYY

SEQ ID NO: 117 - protein, 18 aa, Artificial Sequence
  Synthetic construct
ARETYSQYHYGWFYQGFD

SEQ ID NO: 118 - protein, 8 aa, Artificial Sequence
  Synthetic construct
GGSGSAGG

SEQ ID NO: 119 - protein, 10 aa, Artificial Sequence
  Synthetic construct
GGSGSGGGGS

SEQ ID NO: 120 - protein, 5 aa, Artificial Sequence
  Synthetic construct
GGGSG